(19) United States
(12) Patent Application Publication
(10) Pub. No.: US 2009/0062243 A1
(43) Pub. Date: Mar. 5, 2009

(54) Lupane-Type Triterpenoids Modified at 30-Position and Analogues Thereof

(75) Inventors: Ali Koohang, Plainfield, IL (US); Aye Aye Mar, Tinley Park, IL (US); Erika Szotek, Hanover Park, IL (US); Ze-Qi Xu, Woodridge, IL (US); David A. Eiznhamer, Bloomingdale, IL (US); Michael T. Flavin, Darien, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ADVANCED LIFE SCIENCES, INC., Woodridge, IL (US)

(21) Appl. No.: 12/171,069

(22) Filed: Jul. 10, 2008

Related U.S. Application Data
(60) Provisional application No. 60/963,222, filed on Aug. 3, 2007.

(51) Int. Cl. A61K 31/56; C07J 53/00; A61P 35/04; A61P 29/00; C12N 5/06

(52) U.S. Cl. 514/169; 552/511; 435/375; 514/177

(57) ABSTRACT

The present invention comprises lupine-type triterpenoids that inhibit cell proliferations, in particular cancer and conditions associated with cancer. For example, associated malignancies include ovarian cancer, cervical cancer, breast cancer, colorectal cancer, and glioblastomas, among others. Accordingly, the compounds of the present invention are useful for treating, preventing, and/or inhibiting these diseases. Thus, the present invention also comprising pharmaceutical formulations comprising the compounds and methods of using the compounds and formulations to inhibit cancer and treat, prevent, or inhibit the foregoing diseases.

### CROSS-REFERENCE TO OTHER APPLICATIONS

This application claims the benefit of the filing date, under 35 U.S.C. §119(e), of U.S. Provisional Application Ser. No. 60/963,222, filed 3 Aug. 2007, which is hereby incorporated by reference in its entirety.

### FIELD OF THE INVENTION

This invention relates to the field of inhibitors of cell proliferative conditions. In particular, the invention relates to inhibitors of cancer and conditions associated with cancer.

### BACKGROUND OF THE INVENTION

Lupane-type triterpenoids including betulinic acid (3β-hydroxy-lup-20(29)-en-28-oic acid) (1) and betulin (3β-lup-20(29)-en-3,28-diol, (2) are secondary plant substances with important biological properties, particularly anti-cancer activities in various tumor-derived cell lines such as melanoma, neuroectodermal and leukemia (see, Dzubak et al., Nat. Prod. Rep., 2006, 23, 394-411).

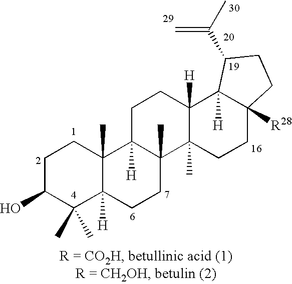

Betulinic acid decreases expression of proangiogenic protein vascular endothelial growth factor (VEGF) and antiapoptotic mitochondrial protein survivin through selective proteosome-dependent targeted inhibition of specificity protein (Sp) transcription factors (see, Chintharlapalli et al., Cancer Res. 2007, 67, 2816-2823). Betulinic acid triggers apoptosis through induction of mitochondrial permeability transition in neuroectodermal tumors (see, Fulda et al., Cancer Res. 1997, 57, 4956-4964; Fulda et al., J. Biol. Chem. 1998, 273, 33942-33948; Ehrhardt et al., Leukemia 2004, 18, 1406-1412; and Eiznhamer et al., IDrugs 2004, 7, 359-373). This mitochondrion-mediated apoptotic process occurs via activation of caspases 3 and 8 and independent of wild-type p53 protein and CD95 ligand/receptor interaction (see, Fulda et al., 1997; Fulda et al., 1998; Ehrhardt et al.; and Eiznhamer et al, supra). Betulinic acid is shown to induce apoptosis by combination with TRAIL (tumor necrosis factor (TNF)-related apoptosis inducing ligand) to enhance the efficacy of TRAIL-induced apoptosis in human tumor cell lines (see, Fulda et al., Oncogene 2004, 23, 7611-7620). Betulinic acid is also used as sensitizer in combination therapy with doxorubicin, VP16, cisplatin, taxol, actinomycin in neuroblastoma cell lines to induce loss of mitochondrial membrane potential and the release of cytochrome c and Smac (second mitochondria-derived activator of caspase) from mitochondria, resulting in activation of caspases and induction of apoptosis (see, Fulda and Debatin, Neoplasia 2005, 7, 162-170).

In the past decade, synthesis and evaluation of new derivatives of betulinic acid has been reported. However, there remains a need in the art for novel anticancer agents, having greater activities and/or reduced side effects.

### SUMMARY OF THE INVENTION

We have embarked upon the design, synthesis and evaluation of triterpenoid derivatives, especially derivatives of betulinic acid (1) and betulin (2). The invention provides compounds, and methods and pharmaceutical compositions comprising the compounds useful for preventing and treating diseases such as cancer. The lupine-type triterpenoids of the invention inhibit cell proliferations, in particular cancer and conditions associated with cancer. For example, associated malignancies include ovarian cancer, cervical cancer, breast cancer, colorectal cancer, and glioblastomas, among others. Accordingly, the compounds of the invention are useful for treating, preventing, and/or inhibiting these diseases. Thus, the invention also comprising pharmaceutical formulations comprising the compounds and methods of using the compounds and formulations to inhibit cancer and treat, prevent, or inhibit the foregoing diseases.

In a first aspect, the invention provides compounds of formula I,

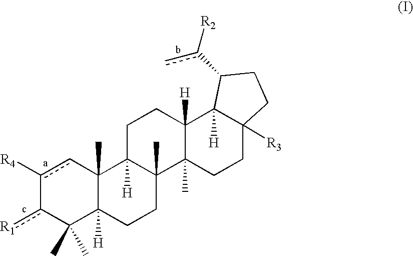

or a pharmaceutically acceptable salt thereof, wherein:

each [symbol] is independently a single or double bond;

when bond c is a double bond, then R₁ is ═O, ═S, ═N—OH, ═N—OR₅, or ═N—OCOR₅; or

when bond c is a single bond, then R₁ is H, halogen, NH₂, OH, SH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₂(R₅), OSO₂(CH₂)ₙX, OSi(R₅)ₘ(R₆)₃₋ₘ, SR₅, SCOR₅, SCO(CH₂)ₙX, SC(O)NHR₅, SC(O)NR₅R₆, NHCOR₅, NHC(O)OR₅, N(R₆)C(O)OR₅, NHC(O)NHR₅, NHC(O)NR₅R₆, N(R₆)C(O)NHR₅, or N(R₆)C(O)NR₅R₆;

R₂ and R₃ are each independently CH₂NH₂, CH₂OH, CH₂SH, CH₂SePh, CHO, CO₂H, CH₂X, CH═N—OH, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NHR₅, CH₂SC(O)NR₅R₆, CH₂NHCOR₅, CH₂NHC(O)OR₅, CH₂NHC(O)NHR₅, CH₂NHC(O)NR₅R₆, CH₂N(R₆)COR₅, CH₂N(R₆)C(O)OR₅, CH₂N(R₆)C(O)NHR₅, CH₂N(R₆)C(O)NR₅R₆, CH═N—OR₅, CH═N—OCOR₅, CH(OR₅)₂, CH(OH)OR₅, CO₂R₅, CO₂NHR₅, or CO₂NR₅R₆;

R₄ is H, halogen, OH, OR₅, CN, CHO, CO₂H, CH(OR₅)₂, CH(OH)OR₅, CO₂R₅, C(O)NHR₅, or C(O)NR₅R₆;

R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein

the aryl comprises phenyl or a polycyclic aryl group such as naphthyl;

the heterocyle comprises
a five or six membered aromatic heterocycle such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, 4-methyl piperazinyl, pyrrolidinyl, imidazolidinyl, imidazolinyl, thiazolidinyl, thiazolinyl, isothiazolidinyl, isothiazolinyl, pyranyl, or morpholinyl;
or a polycyclic heterocycle such as indolyl, benzothienyl, benzofuranyl, isoindolyl, isobenzothienyl, or isobenzofuranyl;

wherein each alkyl, cycloalkyl, aryl, and heterocycle are each optionally substituted with one or more groups (e.g., 1, 2, or 3 group) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl;

or R₅ and R₆ taken together with the carbon atoms to which they are attached form a cyclo(C₃₋₉)alkyl, aryl, or heterocycle, wherein

the aryl comprises a phenyl or a polycyclic aryl group;

the heterocyle comprises
a five or six membered aromatic heterocycle, such as pyridyl, diazinyl, pyrimidinyl, 5-methoxy pyrimidinyl, (1,2,4)triazine-3,5-dione-6-yl, 6-mercaptopyrimidine-4-yl, pyrrolyl, pyrazolyl, imidazolyl, oxazolyl, isoxazolyl, thiazolyl, isothiazolyl, furanyl, or thienyl;
a 3 to 9 membered non-aromatic heterocycle comprising piperazinyl, pyrrolidinyl, imidazolidinyl, imidazolinyl, thiazolidinyl, thiazolinyl, isothiazolidinyl, isothiazolinyl, 4-methyl piperazinyl, pyranyl, or morpholinyl;
or a polycyclic heterocycle such as indolyl, benzothienyl, benzofuranyl, isoindolyl, isobenzothienyl, or isobenzofuranyl;

wherein the cycloalkyl, aryl, and heterocycle are each optionally substituted with one or more groups (e.g., 1, 2, or 3 groups) which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;

each X is independently Y or aryl, heterocycle, or cyclo(C₃₋₉)alkyl, wherein the aryl, heterocycle, or cyclo(C₃₋₉)alkyl are each optionally substituted with one or more Y groups (e.g., 1, 2, or 3 Y groups), wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;

each m is independently 0, 1, 2, or 3;

and each n is independently 1, 2, 3, 4, or 5.

In another aspect, the invention provides pharmaceutical compositions comprising a compound according to the first aspect of the invention and a pharmaceutically acceptable carrier, excipient, or diluent.

In yet another aspect, the invention provides methods for inhibiting cancer in a cell comprising contacting the cell in which inhibition is desired with an effective amount of a compound according to the first aspect of the invention or a composition comprising a compound according to the first aspect of the invention and a pharmaceutically acceptable carrier, excipient, or diluent.

In still another aspect, the invention provides methods of treating a disease comprising administering to a patient a composition comprising a compound according to the first aspect of the invention and a pharmaceutically acceptable carrier, excipient, or diluent. In one embodiment, the disease involves a cell proliferative condition. In another embodiment, the cell proliferative condition is cancer. In still another embodiment, the cancer is melanoma, glioblastoma, ovarian carcinoma, colon carcinoma, and breast carcinoma, or cervical cancer.

In another embodiment, the invention provides methods for inhibiting viruses, bacteria or malaria in a cell comprising contacting the cell in which inhibition is desired with an effective amount of a compound according to the first aspect of the invention or a pharmaceutical composition comprising a compound according to the first aspect of the invention and a pharmaceutically acceptable carrier, excipient, or diluent. In still another embodiment, the invention provides methods for treating inflammation comprising administering to a patient a pharmaceutical composition comprising a compound according to the first aspect of the invention and a pharmaceutically acceptable carrier, excipient, or diluent.

These and other aspects of the present invention will become apparent from the following description of the invention, which are intended to limit neither the spirit nor scope of the invention but are only offered as illustrations of the preferred embodiments of the invention.

### DETAILED DESCRIPTION OF THE INVENTION

In one embodiment of the first aspect, the compounds of formula (I) are subject to the provisos:

when bond a is a single bond, bond b is a double bond, R₁ is [symbol] OH or ═O, R₃ is CO₂H, and R₄ is H, then R₂ is not CH₂OH or CHO;

when bond a is a single bond, bond b is a double bond, R₁ is [symbol] OH, R₃ is CH₂OH, and R₄ is H, then R₂ is not CH₂OH;

when bond a is a single bond, bond b is a double bond, R₁ is [symbol] OH or [symbol] OAc, R₃ is CH₂OH or CH₂OAc, and R₄ is H, then R₂ is not CH₂Br, piperidin-1′-yl, 4-methylpiperidin-1′-yl, 4-benzylpiperidin-1′-yl, 4-methylpiperazin-1′-yl, morpholin-1′-yl, cyclohexylamino, benzylamino, or sec-butylamino;

when bond a is a single bond, bond b is a double bond, R₁ is [symbol] OH or [symbol] OAc, R₃ is CO₂Me, and R₄ is H, then R₂ is not CH₂Br, or 4-methylpiperazin-1′-yl;

when bond a is a single bond, bond b is a double bond, R₁ is [symbol] OAc, R₃ is CH₂OAc, and R₄ is H, then R₂ is not O-methyl-L-valino, dodecylamino, or pentadodecylamino.

In another embodiment of the compounds of formula (I) and any of the preceding embodiments thereof, wherein R₁ is OH, ═O, O—C(O)—CH₃, NH(CH₂)₂OH, NH(CH₂)₂Cl, O-pyranyl, NH(CH₂)₂NHC(O)O-tert-butyl, NH(CH₂)₁₋₂-benzodioxolyl, or NH(CH₂)₁₋₂-phenyl wherein the phenyl is substituted with OH or NH₂.

In another embodiment of the compounds of formula (I) and any of the preceding embodiments thereof, R₂ is CHO, CO₂H, CH₂O-pyranyl, CH₂OH, CH₂SePh, CH₂O Ms, CH₂OAc, CH₂NHCH₂CH₂OH, CH₂NHCH₂CH₂Cl, CH₂NH(CH₂)₂—NHC(O)O-tert-butyl, CH₂NH(CH₂)₁₋₂-benzodioxolyl, or CH₂NH(CH₂)₁₋₂-phenyl wherein the phenyl is substituted with OH or NH₂.

In another embodiment of the compounds of formula (I) and any of the preceding embodiments thereof, R₃ is CHO, CO₂H, CH₂O-pyranyl, CH₂OH, CH₂NHCH₂CH₂OH, CH₂NHCH₂CH₂Cl, CH₂NH(CH₂)₂—NHC(O)O-tert-butyl, CH₂NH(CH₂)₁₋₂-benzodioxolyl, or NH(CH₂)₁₋₂-phenyl, wherein the phenyl is substituted with OH, NH₂, or methylenedioxolyl.

In another embodiment of the compounds of formula (I) and any of the preceding embodiments thereof, wherein R₄ is H, OH, CHO, CO₂H, CN, halogen, OMe.

In another embodiment of the compounds of formula (I) and any of the preceding embodiments thereof, each X is F, Cl, Br, I, CN, N₃, NHOH, ═NOH, NH₂, OH, SH, NHR₅, NR₅R₆, OR₅, SR₅, CO₂H, CO₂R₅, SO₃H₂, or SO₃R₅.

The chemistries described in the invention represent methods for the syntheses of compounds of the general structural formula I and, therefore, the present invention relates to compounds, compositions and methods for preventing and treating diseases, for the prevention and inhibition of tumor growth and for the treatment of malignant tumors such as melanoma, glioblastoma, ovarian carcinoma, colon carcinoma, and breast carcinoma.

### DEFINITIONS

As used in the present specification, the following words and phrases are generally intended to have the meanings as set forth below, except to the extent that the context in which they are used indicates otherwise or they are expressly defined to mean something different.

The symbol “—” means a single bond, “═” means a double bond, “≡” means a triple bond, [symbol] means a single or double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

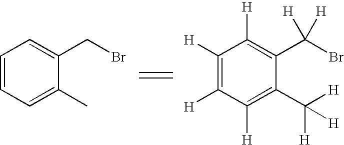

“Alkyl” is intended to include linear, branched, or cyclic hydrocarbon structures and combinations thereof, inclusively. For example, “C₆ alkyl” may refer to an n-hexyl, iso-hexyl, cyclobutylethyl, and the like. Lower alkyl refers to alkyl groups of from one to six carbon atoms. Examples of lower alkyl groups include methyl, ethyl, propyl, isopropyl, butyl, s-butyl, t-butyl, isobutyl, pentyl, hexyl and the like. Higher alkyl refers to alkyl groups containing more that eight carbon atoms, for example, eight to twenty carbon atoms. Exemplary alkyl groups are those of C₂₀ or below, or C₁₂ or below, or C₈ or below. Cycloalkyl is a subset of alkyl and includes cyclic hydrocarbon groups of from three to thirteen carbon atoms, as further defined below. In this application, alkyl refers to alkanyl, alkenyl, and alkynyl residues (and combinations thereof); it is intended to include cyclohexylmethyl, vinyl, allyl, isoprenyl, and the like. Thus when an alkyl residue having a specific number of carbons is named, all geometric isomers having that number of carbons are intended to be encompassed; thus, for example, either “butyl” or “C₄ alkyl” is meant to include n-butyl, sec-butyl, isobutyl, t-butyl, isobutenyl and but-2-ynyl groups; and for example, “propyl” or “C₃ alkyl” each include n-propyl, propenyl, and isopropyl. Alkyl also includes unsaturated hydrocarbon groups, such as alkenyl and alkynyl groups each having one or more carbon-carbon double or triple bonds, respectively.

“Alkoxy” or “alkoxyl” refers to the group —O-alkyl, for example including from one to eight carbon atoms of a straight, branched, cyclic configuration, unsaturated chains, and combinations thereof attached to the parent structure through an oxygen atom. Examples include methoxy, ethoxy, propoxy, isopropoxy, cyclopropyloxy, cyclohexyloxy and the like. Lower-alkoxy refers to groups containing one to six carbons.

“Aryl” refers to aromatic six- to fourteen-membered carbocyclic ring, and includes mono-, bicyclic or polycyclic groups, for example, benzene, naphthalene, acenaphthylene, anthracene, indane, tetralin, fluorene and the like. Aryl as substituents includes univalent or polyvalent substituents. As univalent substituents, the aforementioned ring examples are named, phenyl, naphthyl, acenaphthyl, anthracenyl, indanyl, tetralinyl, and fluorenyl. “Polycyclic aryl” as used herein refers to an aryl ring fused to at least a second aryl ring. Examples of polycyclic aryl include, but are not limited to, naphthyl, anthracenyl, acenaphthylenyl, and phenanthrenyl.

When a group is referred to as “arylalkyl”, such as “aryl-C₁-C₈ alkyl”, an aryl moiety is attached to a parent structure via an alkylene group. Examples include benzyl, phenethyl, and the like. Both the aryl and the corresponding alkylene portion of a “C₁-C₆ alkyl-aryl” group may be optionally substituted, as defined herein.

In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Cycloalkyl” refers to a “cycloalkanyl”, “cycloalkenyl”, and “cycloalkynyl” groups, where “cycloalkanyl” refers to fully saturated hydrocarbon rings; “cycloalkenyl” refers to non-aromatic hydrocarbon rings containing at least one carbon-carbon double bond; and “cycloalkynyl” refers to non-aromatic hydrocarbon rings containing at least one carbon-carbon triple bond. Each cycloalkyl group can be a monocyclic, fused or bridged bicyclic, fused or bridged tricyclic, fused or bridged polycyclic hydrocarbon group comprising 3 to 14 carbon atoms in the cycloalkyl ring, where the cycloalkyl can be saturated or unsaturated with one or more carbon-carbon double and/or triple bonds between consecutive ring atoms. Examples of cycloalkynyl group include, but are not limited to, cyclopropyl, cyclobutyl, cyclopentyl, cyclohexyl, cyclooctyl, decahydronaphthalenyl, bicyclo[2.2.1]heptanyl, adamantyl, and bicyclo[2.2.2]octanyl. Examples of cycloalkenyl group include, but are not limited to, cyclopentenyl, cyclohexenyl, cyclooctenyl, cyclooctadienyl, octahydronaphthalenyl, norbornenyl, and bicyclo[2.2.2]octenyl. Examples of cycloalkynyl group include, but are not limited to, cyclooctynyl and cyclodecenyl.

“Fused-polycyclic” or “fused ring system” refers to a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have spiro rings attached thereto via a single ring atom of the fused-polycyclic.

“Halogen” or “halo” refers to fluorine, chlorine, bromine or iodine. “Haloalkyl” and “haloaryl” refer generically to alkyl and aryl groups that are substituted with one or more halogens, respectively. Thus, “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is within the scope of dihaloaryl. The phrase “mono- to per-halogenated” when combined with another group refers to groups wherein one hydrogen, more than one hydrogen, or all hydrogens are replaced with a halo. For example, a “mono- to per-halogenated methyl” would encompass groups such as —CH₂F, —CHCl₂ or —CF₃.

“Heterocycle” or “heterocyclyl” refers to a stable three- to fifteen-membered ring substituent that consists of carbon atoms and from one to five heteroatoms selected from the group consisting of nitrogen, phosphorus, oxygen and sulfur. A heterocycle includes an aromatic heterocyclyl group (i.e., heteroaryl). The heterocyclyl substituent may be a monocyclic, bicyclic or tricyclic ring system, which includes fused or bridged ring systems as well as spirocyclic systems; and the nitrogen, phosphorus, carbon or sulfur atoms in the heterocyclyl group may be optionally oxidized to various oxidation states. In a specific example, the group —S(O)₀₋₂—, refers to —S-(sulfide), —S(O)— (sulfoxide), and —SO₂— (sulfone). For convenience, nitrogens, particularly but not exclusively, those defined as annular aromatic nitrogens, are meant to include their corresponding N-oxide form, although not explicitly defined as such in a particular example. Thus, for a compound of the invention having, for example, a pyridyl ring; the corresponding pyridyl-N-oxide is meant to be included as another compound of the invention. In addition, annular nitrogen atoms may be optionally quaternized; and the ring substituent may be partially or fully saturated or aromatic. Examples of heterocyclyl groups include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazolyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, dioxaphospholanyl, and oxadiazolyl.

Preferred heterocyclyls include, but are not limited to, acridinyl, azocinyl, benzimidazolyl, benzofuranyl, benzothiophenyl, benzoxazolyl, benzthiazolyl, benztriazolyl, pyridotriazolyl, benzisoxazolyl, benzisothiazolyl, benzimidazolonyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,3,4-triazolyl, and xanthenyl.

“Polycyclic heterocycle” as used herein refers to a heterocycle fused to at least one other aryl or heterocyclyl ring, as defined herein. Examples of bicyclic heterocycles include, but are not limited to, indolyl, benzimidazolyl, benzofuranyl, benzothiophenyl, benzoxazolyl, benzthiazolyl, benztriazolyl, pyridotriazolyl, benzisoxazolyl, benzisothiazolyl, and carbazolyl,

When a group is referred to as “heterocyclylalkyl” such as “heterocyclyl-C₁-C₈ alkyl” a heterocycle moiety is attached to a parent structure via an alkylene group. Examples include pyrid-2-ylmethyl, morpholin-4-ylmethyl, piperidin-1-ylmethyl, and the like. Both the heterocycle and the corresponding alkylene portion of a “C₁-C₆ alkyl-heterocyclyl” group may be optionally substituted, as defined herein.

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted aryl-C₁₋₈ alkyl,” both the “C₁₋₈ alkyl” portion and the “aryl” portion of the molecule may or may not be substituted. A list of exemplary optional substitutions is presented below in the definition of “substituted.” “Substituted” alkyl, aryl, and heterocyclyl, refer respectively to alkyl, aryl, and heterocyclyl groups having one or more (for example up to about five, in another example, up to about three) hydrogen atoms replaced by a substituent independently selected from the group consisting of alkyl (for example, fluoromethyl), aryl (for example, 4-hydroxyphenyl), arylalkyl (for example, 1-phenyl-ethyl), heterocyclylalkyl (for example, 1-pyridin-3-yl-ethyl), heterocyclyl (for example, 5-chloro-pyridin-3-yl or 1-methyl-piperidin-4-yl), alkoxy, alkylenedioxy (for example methylenedioxy), amino (for example, alkylamino and dialkylamino), amidino, aryloxy (for example, phenoxy), arylalkyloxy (for example, benzyloxy), carboxy (—CO₂H), carboalkoxy (that is, acyloxy or —OC(═O)R), carboxyalkyl (that is, esters or —CO₂R), carboxamido, benzyloxycarbonylamino (CBZ-amino), cyano, acyl, halogen, hydroxy, nitro, sulfanyl, sulfinyl, sulfonyl, thiol, halogen, hydroxy, oxo, carbamyl, acylamino, and sulfonamido. Each substituent of a substituted group can be optionally substituted, but these further optional substituents themselves are not further substituted. Thus, an optionally substituted moiety is one that may or may not have one or more substituents, and each of the substituents may or may not have one or more substituents.

Some of the compounds of the invention may have imino, amino, oxo or hydroxy substituents off aromatic heterocyclyl systems. For purposes of this disclosure, it is understood that such imino, amino, oxo or hydroxy substituents may exist in their corresponding tautomeric form, i.e., amino, imino, hydroxy or oxo, respectively.

Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS).

The compounds of the invention, or their pharmaceutically acceptable salts, may have asymmetric carbon atoms, oxidized sulfur atoms or quaternized nitrogen atoms in their structure.

The compounds of the invention and their pharmaceutically acceptable salts may exist as any and all possible stereoisomers, geometric isomers, enantiomers, diastereomers and anomers. All such single stereoisomers, racemates and mixtures thereof, and geometric isomers are intended to be within the scope of this invention.

The description of the invention herein should be construed in congruity with the laws and principals of chemical bonding. It is assumed that when considering generic descriptions of compounds of the invention for the purpose of constructing a compound, such construction results in the creation of a stable structure. That is, one of ordinary skill in the art would recognize that theoretically some constructs which would not normally be considered as stable compounds (that is, sterically practical and/or synthetically feasible, supra).

When a particular group with its bonding structure is denoted as being bonded to two partners; that is, a divalent group, for example, —OCH₂—, then it is understood that either of the two partners may be bound to the particular group at one end, and the other partner is necessarily bound to the other end of the particular group, unless stated explicitly otherwise. Stated another way, divalent groups are not to be construed as limited to the depicted orientation, for example “—OCH₂—” is meant to mean not only “—OCH₂—” as drawn, but also “—CH₂O—.”

In addition to the preferred embodiments recited hereinabove, also preferred are embodiments comprising combinations of preferred embodiments.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to their knowledge and to this disclosure.

“Cancer” refers to cellular-proliferative disease states, including but not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinorna, glucagonoma, gastrinoma, carcinoid tumors, vipoma), small bowel (adenocarcinorna, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis defomians), meninges (meningioma, meningiosarcoma, gliomatosis), brain (astrocytoma, medulloblastoma, glioma, ependymoma, germinoma [pinealoma], glioblastorna multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, SertoliLeydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

“Pharmaceutically acceptable salt” include acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge et al., J. Pharm. Sci. 1977, 66, 1-19 which is incorporated herein by reference.)

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover compounds made either using standard organic synthetic techniques, including combinatorial chemistry or by biological methods, such as bacterial digestion, metabolism, enzymatic conversion, and the like.

“Treating” or “treatment” as used herein covers the treatment of a disease-state in a human, which disease-state is characterized by abnormal cellular proliferation, and invasion and includes at least one of: (i) preventing the disease-state from occurring in a human, in particular, when such human is predisposed to the disease-state but has not yet been diagnosed as having it; (ii) inhibiting the disease-state, i.e., arresting its development; and (iii) relieving the disease-state, i.e., causing regression of the disease-state. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

### General Administration

In the second aspect, the invention provides pharmaceutical compositions comprising compounds according to the first aspect of the invention and a pharmaceutically acceptable carrier, excipient, or diluent. In certain other preferred embodiments, administration may preferably be by the oral route. Administration of the compounds of the invention, or their pharmaceutically acceptable salts, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, rectally, or via urethral, ocular intratumoral and irrigation method, in the form of solid, semi-solid, lyophilized powder, or liquid dosage forms, such as for example, tablets, suppositories, pills, soft elastic and hard gelatin capsules, powders, solutions, suspensions, or aerosols, or the like, preferably in unit dosage forms suitable for simple administration of precise dosages.

The compositions will include a conventional pharmaceutical carrier or excipient and a compound of the invention as the/an active agent, and, in addition, may include other medicinal agents, pharmaceutical agents, carriers, adjuvants, etc. Compositions of the invention may be used in combination with anticancer or other agents that are generally administered to a patient being treated for cancer. Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc. The dosage form can be designed as a sustained release or timed release.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), dextrose, mannitol, polyvinylpyrrolidone, gelatin, hydroxycellulose, acacia, suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants. The liquid formulation can be buffered, isotonic solution.

One preferable route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see, Remington's Pharmaceutical Sciences, 18th Ed., (Mack Publishing Company, Easton, Pa., 1990). The composition to be administered will, in any event, contain a therapeutically effective amount of a compound of the invention, or a pharmaceutically acceptable salt thereof, for treatment of a disease-state in accordance with the teachings of this invention.

The compounds of the invention, or their pharmaceutically acceptable salts, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of the present invention can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

### Synthetic Procedures

Direct oxidation of the THP-protected hydroxyl ketone 4, prepared from betulin (2) via compound 3, by PhSeCl in pyridine and CH₂Cl₂ followed by the treatment with 30% H₂O₂, led to the formation of 30-hydroxy ketone 5. When the H₂O₂ oxidation step was omitted from the sequence, the intermediate of allylic phenylselenide 6 was isolated. Deprotection of THP group from 5 and 6 furnished the 3-keto compound 7, which was then reduced by NaBH₄ to afford betulin derivatives modified at 30-position (8) (Scheme 1).

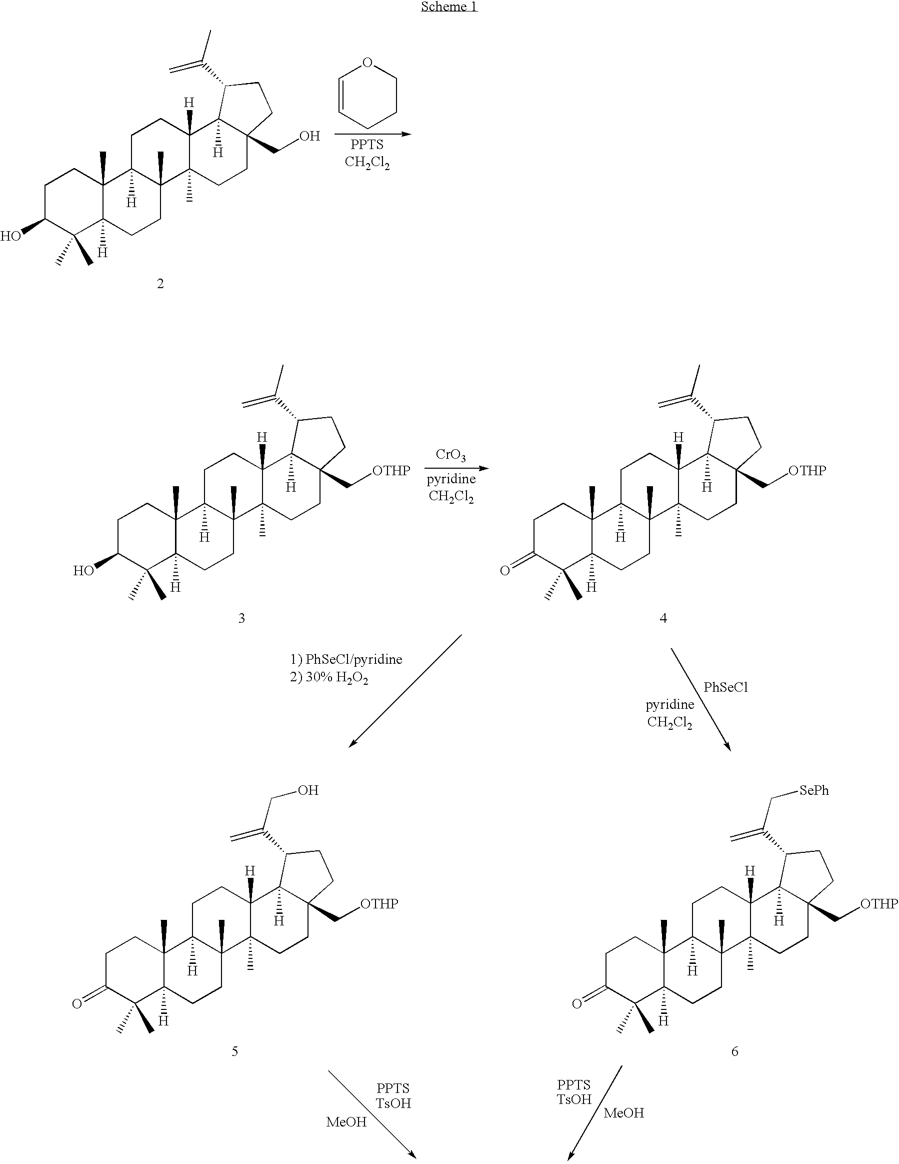

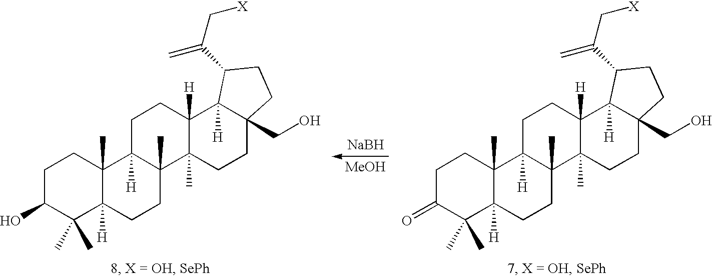

The 30-hydroxy group in 5 can be further modified to yield novel lupane-type pentacyclic triterpenoids. For example, selective oxidation of 5 with CrO₃·2pyridine complex provided α,β-unsaturated aldehyde 9 which was converted to the dimethyl acetal derivative 10 upon removal of the 28-THP protecting group (PPTS, TsOH, MeOH) (Scheme 2). When the keto alcohol 5 was subjected to Jones oxidation, the bis-carboxylic acid 11 was obtained. The over-oxidation at the 28-position was attributed to the instability of THP protecting group under the strong acidic conditions.

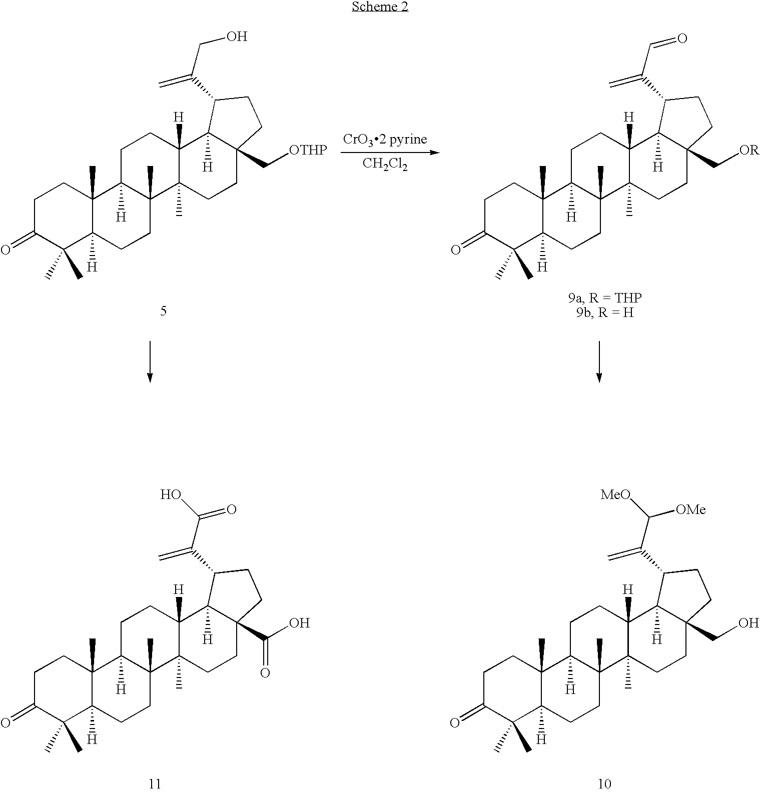

When an acid-stable acetyl group was used as the protecting group, in stead of THP, the over-oxidation of the 28-OH was circumvented. Thus, Jones oxidation of 12 provided the mono acid 13a as the major product, along with the partially oxidized product, aldehyde 14a (Scheme 3). Triterpenoids 13a and 14a underwent basic hydrolysis to afford 28-hydroxy analogues 13b and 14b respectively. The keto hydroxy acid 13b was reduced by NaBH₄ to afford the corresponding carboxy acid diol 15.

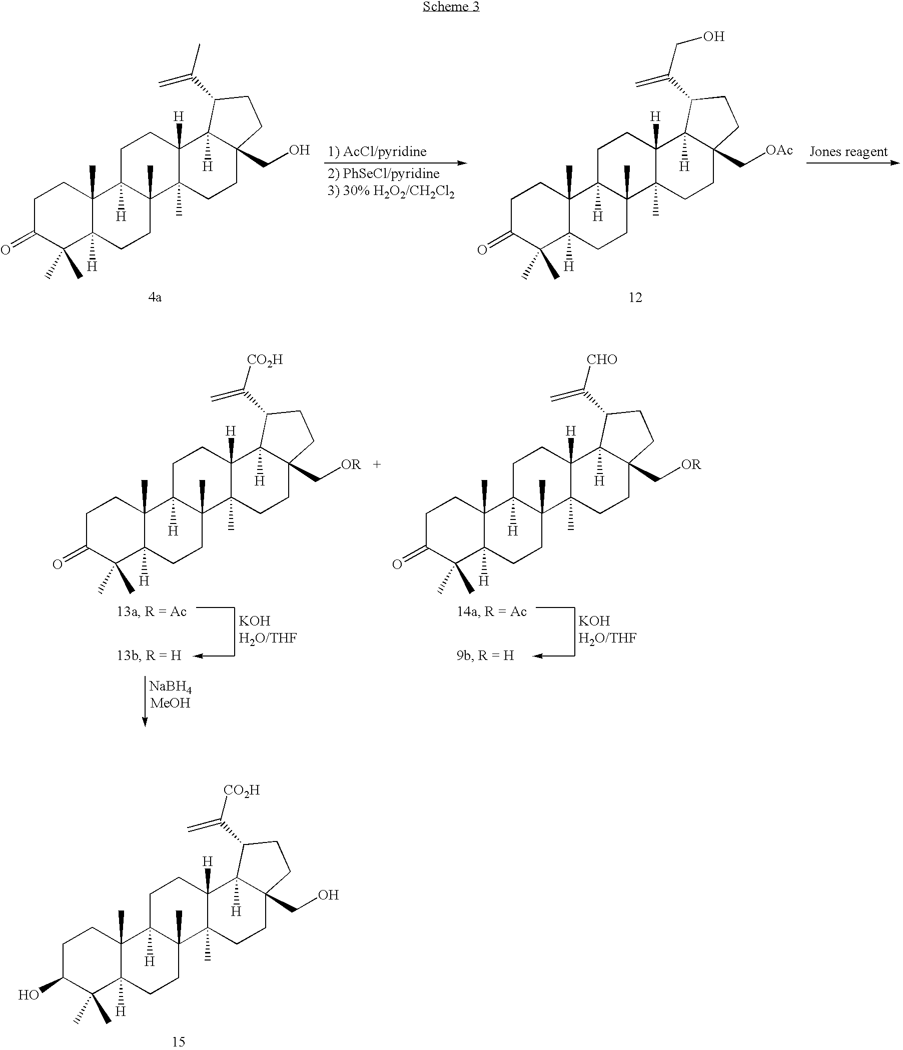

In order to synthesize 30-hydroxybetulinic acid derivatives, the 30-OH was protected by an acetyl group as described above and the 28-OH was freed upon the removal of THP group. The resulting 30-O-Acetyl-28-hydroxy compound 16 was then oxidized by Jones reagent to provide 30-O-acetyl betulonic acid 17a as the major product, along with the partially oxidized aldehyde 18a (Scheme 4). The acetyl group in both 17a and 18a were readily hydrolyzed under the basic conditions, affording the 30-OH derivatives 17b and 18b. The keto group in 17a and 17b were reduced by NaBH₄, respectively, to the corresponding 30-acetoxy and 30-hydroxy betulinic acid 19a and 19b. Oxidation of 18b by CrO₃/pyridine resulted in the bis-aldehyde 20 (Scheme 4).

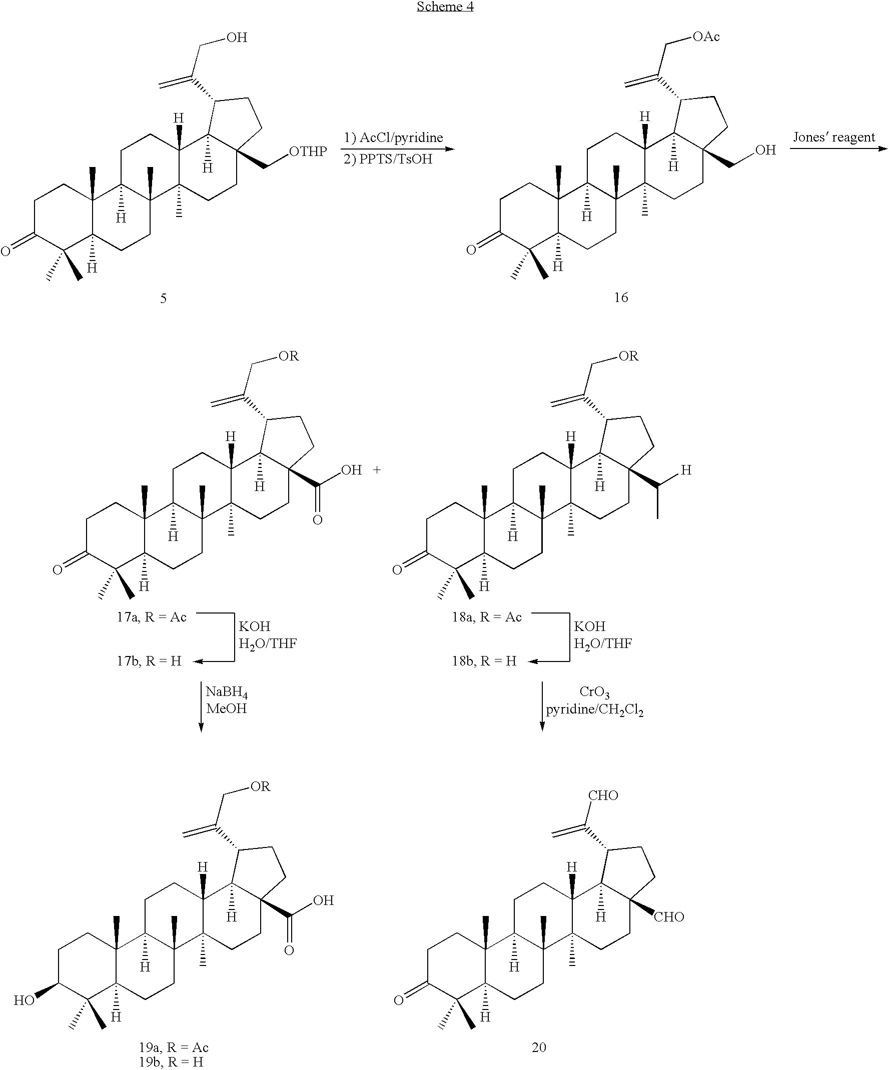

The 30-OH group in betulone 5, 17b and 18b could be substituted with other functional groups with increased potency, enhanced solubility profile and biopharmaceutical properties. For example, 30-aza derivatives 22 were synthesized by S_{N}2 displacement of the mesylate 21 with various amines. Reduction of 22 furnished 23 (Scheme 5).

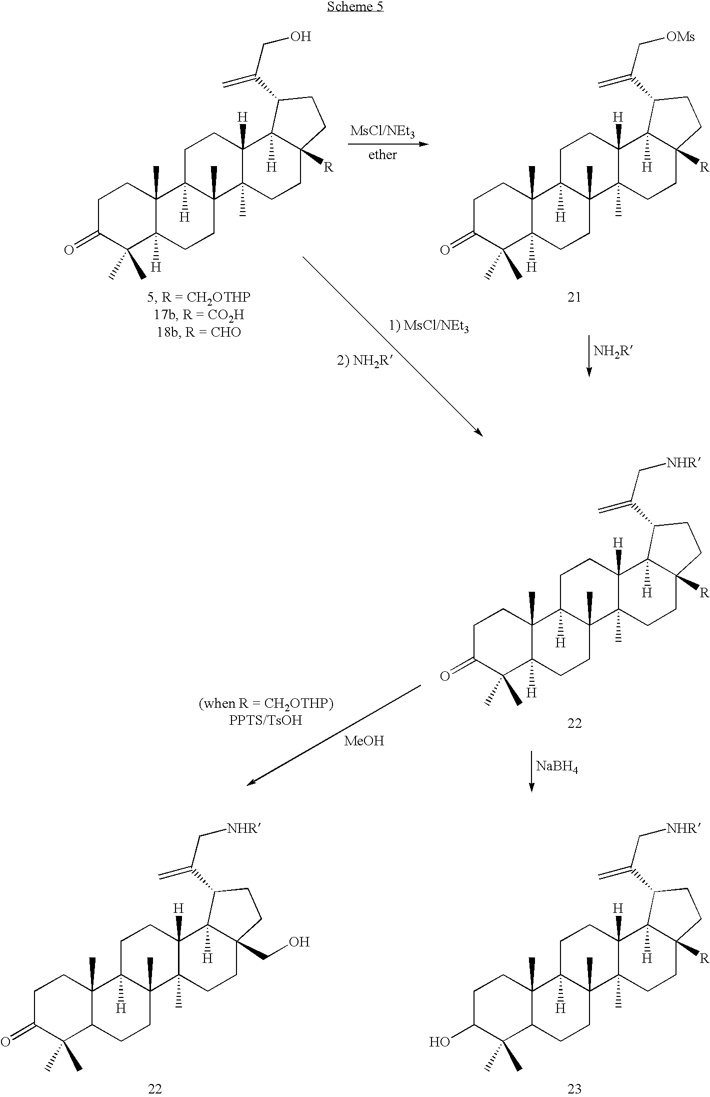

Furthermore, the 3-keto, 28- and 30-aldehyde groups in compounds such as 5-7, 9-14, 16-18 and 21-23 offers an additional site for modification. For instance, reductive amination with various amines led to the formation of the corresponding amino analogues 24-26 (Scheme 6).

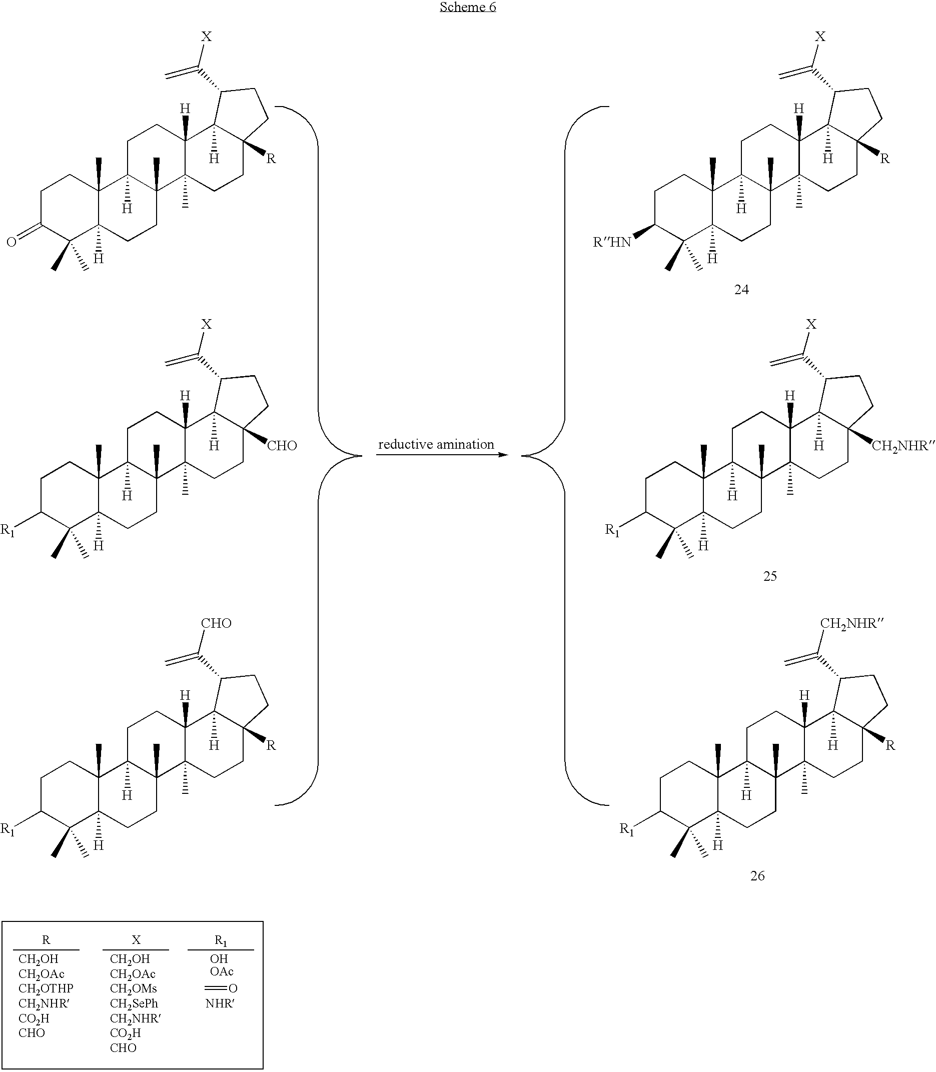

In addition, dehydrogenation of 4 by PhSeCl and 30% aqueous H₂O₂ (Scheme 7), afforded a mixture of 1,2-en-3-one 27 and 1,2-en-3-one-30-hydroxy 28. Both enones 27 and 28 were isolated and characterized as 1:1 mixtures with their 1,2-saturated analogues by ¹H NMR analysis. De-protection of enones 27 and 28 provided the corresponding keto alcohol 29 and keto diol 30, respectively, which were again inseparable mixtures with their 1,2-saturated analogues.

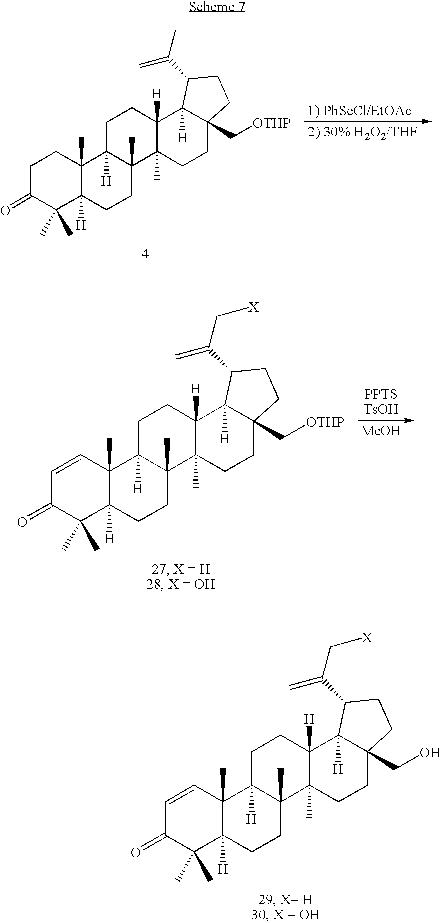

A similar dehydrogenation is applicable to triterpenoids modified at the 2-position. For example, the α-formyl analogue 31, prepared by formylation of 4 (NaOMe, HCO₂Et, MeOH), was dehydrogenated by α-selenation and H₂O₂ to furnish the corresponding α,β-unsaturated 30-OH aldehyde 32, along with compound 33 (Scheme 8).

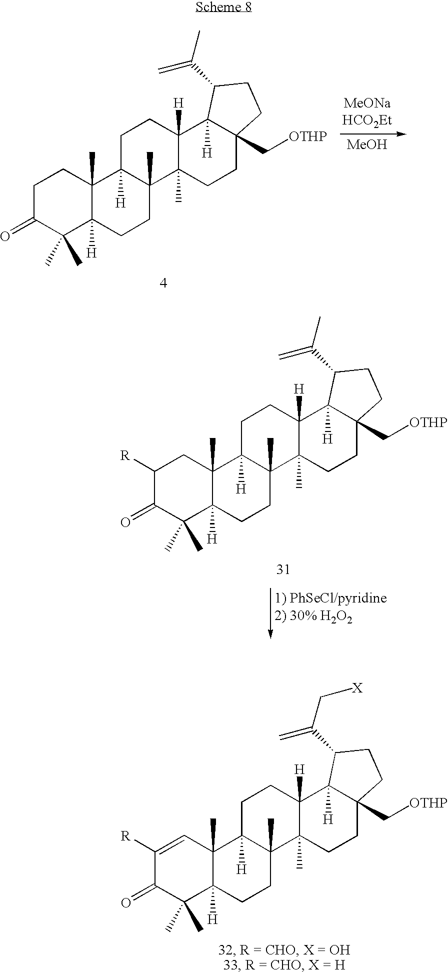

However, when benzeneseleninic anhydride was used and the reaction proceeded in chlorobenzene under refluxing condition, 1,2-en-3-one-30-aldehyde 34 was obtained, along with the corresponding deprotected aldehyde 35 (Scheme 9).

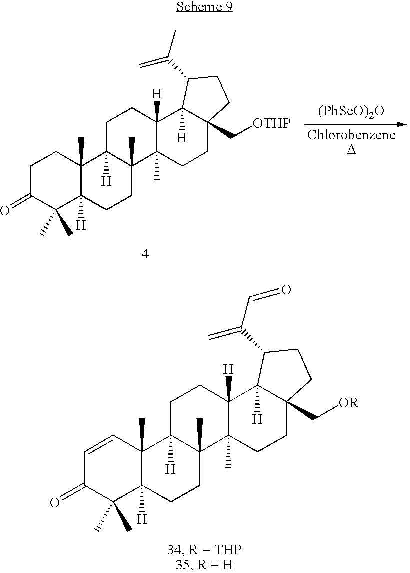

The double bond at 20(29) position in the compounds described above can be hydrogenated to furnish the corresponding saturated derivatives 36 (Scheme 10).

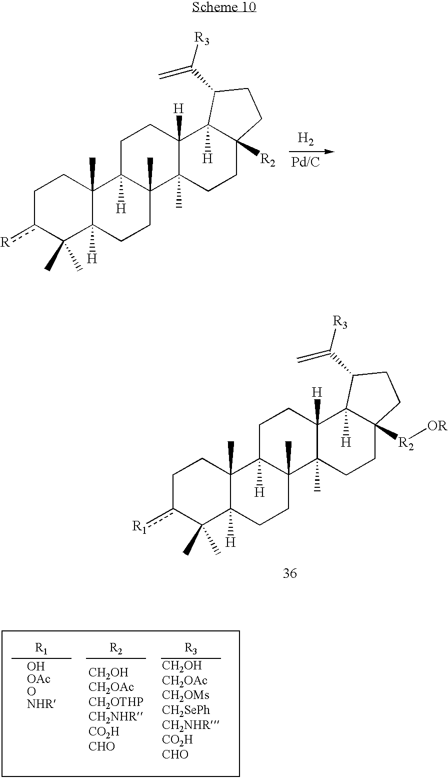

### EXAMPLES

### Example 1

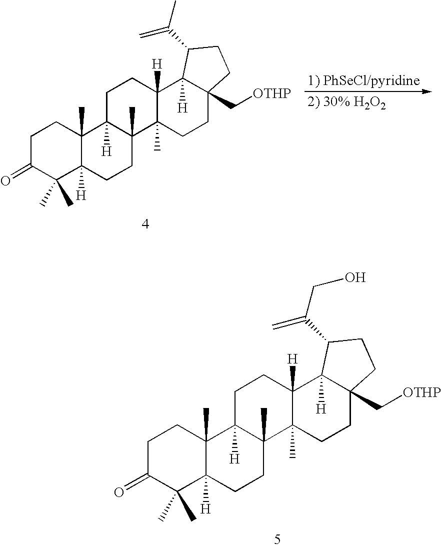

To a solution of benzeneselenenyl chloride (0.80 g, 4.19 mmol) in CH₂Cl₂ (60 mL), while stirring at 0° C. under N₂, was added pyridine (0.53 mL, 6.51 mmol). The dark orange solution turned to light yellow which was stirred for 15 min at room temperature whereupon a solution of compound 4 (2.0 g, 3.81 mmol) in CH₂Cl₂ (40 mL) was added. After 1 h at room temperature the reaction mixture was quenched with 10% HCl (100 mL). The organic layer was separated and cooled at 0° C. into which 30% aqueous H₂O₂ (1 mL, 9.8 mmol) was added dropwise. After 1 h, the reaction mixture was warmed up to room temperature and washed with saturated NaHCO₃ (100 mL). The organic layer was separated, dried (Na₂SO₄) and concentrated under reduced pressure to a foamy residue (3.0 g) which was purified by silica gel column chromatography (5-50% EtOAc/hexanes) to give 618 mg (31% yield) of the recovered starting material 4 and 912 mg (44% yield) of 5 as a mixture of diastereomers. ¹H NMR (500 MHz, CDCl₃) δ 0.90-2.24 (m), 0.92 (s), 0.98 (s), 1.023 (s), 1.024 (s), 1.05 (s), 1.07 (s), 2.41 (m), 2.47 (m), 2.98 (d, J=9.5 Hz), 3.36 (d, J=10 Hz), 3.51 (m), 3.82 (m), 3.91 (d, J=8.5 Hz), 4.11 (AB quartet), 4.54 (t, J=3.5 Hz), 4.58 (t, J=3.5 Hz), 4.90 (s), 4.94 (d, J=1.5 Hz).

### Example 2

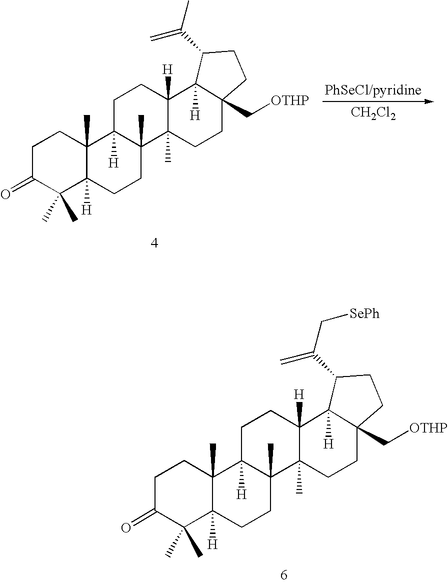

To a solution of benzeneselenenyl chloride (87 mg, 0.45 mmol) in CH₂Cl₂ (6 mL), while stirring at 0° C. under N₂, was added pyridine (0.1 mL, 1.23 mmol). The dark orange solution turned to light yellow which was stirred for 0.5 h at 0° C., whereupon a solution of compound 4 (207 mg, 0.39 mmol) in CH₂Cl₂ (3 mL) was added. The homogeneous mixture was warmed up to room temperature and after 3 h, the reaction mixture was quenched with 10% HCl (10 mL). The organic layer was separated and washed successively with saturated NaHCO₃ (10 mL) and saturated NaCl (10 mL). After dried (Na₂SO₄), concentration under reduced pressure yielded a dark yellow crude product. Purification by silica gel column chromatography using gradient EtOAc/hexanes (5-20%) provided 218 mg (81% yield) of a white fluffy powder 6 as a mixture of diasteromers: ¹H NMR (500 MHz, CDCl₃) δ 0.80-2.3 (m), 0.92 (s), 0.97 (s), 1.024 (s), 1.026 (s), 1.05 (s), 1.07 (s), 2.41 (m), 2.49 (m), 2.96 (d, J=9 Hz), 3.36 (d, J=9.5 Hz), 3.50 (d, J=9.5 Hz), 3.57 (m), 3.82 (m), 3.91 (d, J=9.5 Hz), 4.54 (t, J=3.5 Hz), 4.59 (t, J=3.5 Hz), 4.83 (s), 7.25 (m), 7.48 (m).

### Example 3

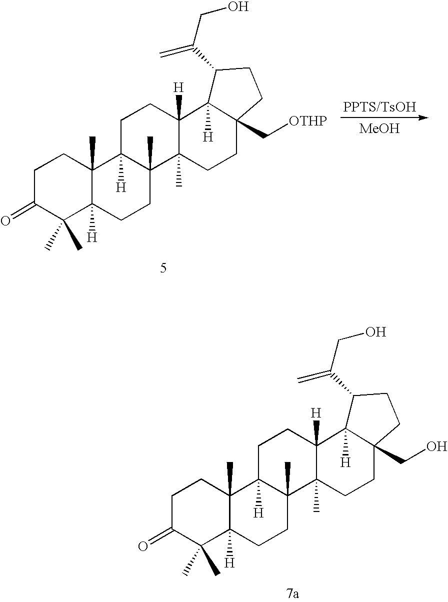

To a solution of 5 (51 mg, 0.09 mmol) in MeOH (2 mL), while stirring at rt, were added in succession pyridinium p-toluenesulfonate (20 mg, 0.08 mmol) and p-toluenesulfonic acid monohydrate (20 mg, 0.11 mmol). After 16 h, the reaction mixture was diluted with EtOAc (5 mL) and washed with saturated NaHCO₃ (5 mL). The organic layer was separated, dried (Na₂SO₄) and concentrated under reduced pressure. Purification by SiO₂ column chromatography with gradient elution (20-60% EtOAc/hexanes) afforded 35 mg (81% yield) of 7a as white solid. ¹H NMR (500 MHz, CDCl₃) δ 0.8-2.0 (m), 0.92 (s), 0.99 (s), 1.02 (s), 1.06 (s), 1.07 (s), 2.10 (m), 2.31 (m), 2.41 (m), 2.47 (m), 3.33 (m), 3.79 (m), 4.12 (m), 4.90 (s), 4.96 (m).

### Example 4

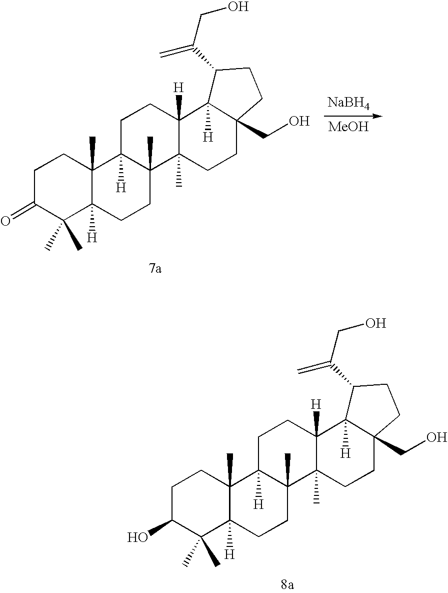

To a solution of 7a (47 mg, 0.10 mmol) in MeOH (3 mL), while stirring at room temperature under N₂, was added NaBH₄ (8 mg, 0.21 mmol). After 2 h, the reaction mixture was diluted with EtOAc (10 mL) and washed successively with 1 N HCl (10 mL), saturated NaHCO₃ (10 mL) and brine (10 mL). The organic layer was separated, dried (Na₂SO₄) and concentrated under reduced pressure to a solid which was purified by gradient SiO₂ column chromatography (30-50% EtOAc/hexanes) to afford 32 mg (68% yield) of 8a as white solid. ¹H NMR (500 MHz, CD₃OD) δ 0.7-1.74 (m), 0.75 (s), 0.86 (s), 0.95 (s), 1.01 (s), 1.07 (s), 1.79 (m), 1.90 (dd, J=8.5, 12 Hz), 1.96 (m), 2.11 (m), 2.30 (m), 3.11 (dd, J=4.5, 11 Hz), 3.27 (d, J=11 Hz), 3.73 (d, J=11 Hz), 4.03 (m), 4.85 (m), 4.94 (m). ¹³C NMR (500 MHz, CDCl₃) δ 15.3, 16.2, 16.7, 16.8, 19.6, 22.2, 28.14, 28.18, 28.3, 28.7, 30.5, 32.9, 35.0, 35.6, 38.4, 38.7, 40.1, 40.2, 42.3, 43.9, 44.9, 49.1, 50.7, 52.0, 56.9, 60.2, 65.2, 79.8, 107.2, 156.1.

### Example 5

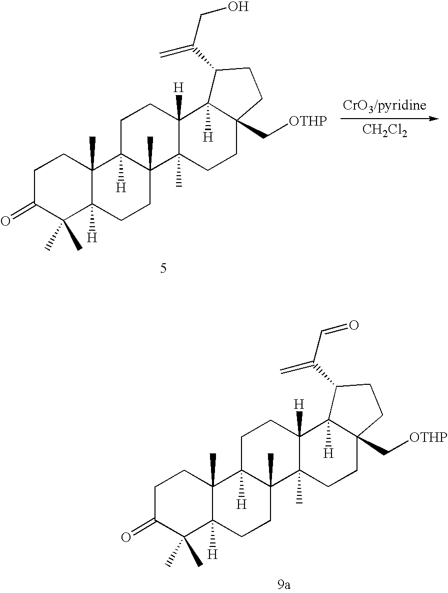

To a solution of pyridine (0.36 mL, 4.43 mol) in CH₂Cl₂ (4 mL), while stirring at room temperature under N₂, was added CrO₃ (221 mg, 2.21 mol). The resulting dark brown suspension was stirred for an additional hour and it was then cooled to 0° C., into which was added dropwise a solution of 5 (200 mg, 0.37 mmol) in CH₂Cl₂ (2 mL). After 1 h, the suspension was decanted and the solids were washed with additional CH₂Cl₂ (10 mL). The combined organic solutions were concentrated under reduced pressure to a dark brown solid which was purified by silica gel column chromatography (0-20% EtOAc/hexanes) to afford 136 mg (68% yield) of a white foam 9a as mixture of diastereomers. ¹H NMR (500 MHz, CDCl₃) δ 0.8-2.3 (m), 0.91 (s), 0.959 (s), 0.961 (s), 1.019 (s), 1.021 (s), 1.04 (s), 1.059 (s), 1.067 (s), 2.38 (m), 2.47 (m), 2.82 (m), 3.04 (d, J=9.5 Hz), 3.37 (d, J=10 Hz), 3.53 (m), 3.56 (d, J=9.5 Hz), 3.84 (m), 3.92 (d, J=10 Hz), 4.53 (t, J=3 Hz), 4.59 (t, J=3 Hz), 5.91 (s), 6.27 (s), 9.51 (d, J=1 Hz).

### Example 6

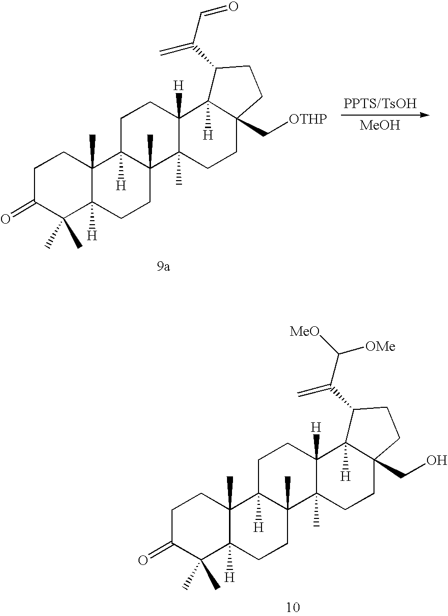

To a solution of 9a (39 mg, 0.07 mmol) in MeOH (2 mL), while stirring at rt, were added in succession pyridinium p-toluenesulfonate (18 mg, 0.07 mmol) and p-toluenesulfonic acid monohydrate (14 mg, 0.07 mmol). After 16 h, the reaction mixture was diluted with EtOAc (10 mL) and washed with saturated NaHCO₃ (10 mL). The organic layer was separated, dried (Na₂SO₄) and concentrated under reduced pressure. Purification by SiO₂ column chromatography with gradient elution (20-50% EtOAc/hexanes) afforded 24 mg (75% yield) of 10 as white solid. ¹H NMR (500 MHz, CDCl₃) δ 0.8-2.6 (m), 0.92 (s), 1.00 (s), 1.02 (s), 1.06 (s), 1.07 (s), 3.30 (s), 3.31 (s), 3.33 (m), 3.78 (m), 4.54 (s), 5.05 (s), 5.09 (s). Note: This sample contains about 15% of the free aldehyde by ¹H NMR integration analysis. The aldehyde diagnostic peaks include the following chemical shifts: 5.93 (s), 6.28 (s), 9.51 (s).

### Example 7

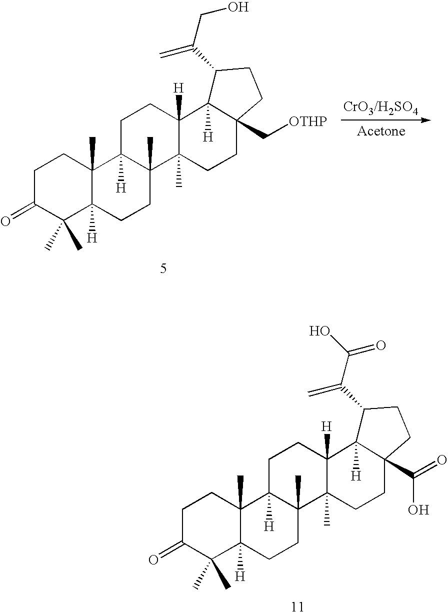

To a solution of 5 (200 mg, 0.37 mmol) in acetone (4 mL), while stirring at 0° C. under N₂, was added dropwise the freshly prepared Jones reagent (0.80 mL, 1.96 M, 1.57 mmol). When the purple-brown precipitates were formed, the reaction mixture was warmed up to room temperature and stirring continued. The reaction was monitored by TLC analysis. After 5 h, the starting material was still present. The reaction mixture was then cooled to 0° C. and another aliquot of the Jones reagent (0.4 mL, 0.78 mmol) was added. After stirring for another hour at rt, no starting material was observed by TLC analysis. The dark brown reaction mixture was partitioned between EtOAc (10 mL) and aqueous Na₂S₂O₅ (10 mL). The organic layer was separated, dried (Na₂SO₄) and concentrated under reduced pressure to a clear oil which was purified by SiO₂ silica gel column chromatography (0-50% EtOAc/hexanes) to afford 136 mg of 11 (76% yield) as a white powder. ¹H NMR (CDCl₃, 500 MHz) δ 0.8-2.6 (m), 0.92 (s), 0.97 (s), 1.01 (s), 1.06 (s), 3.45 (m), 5.7 (s), 6.27 (s); Negative ESI-MS, m/e 483.5 (M−H)⁺.

### Example 8

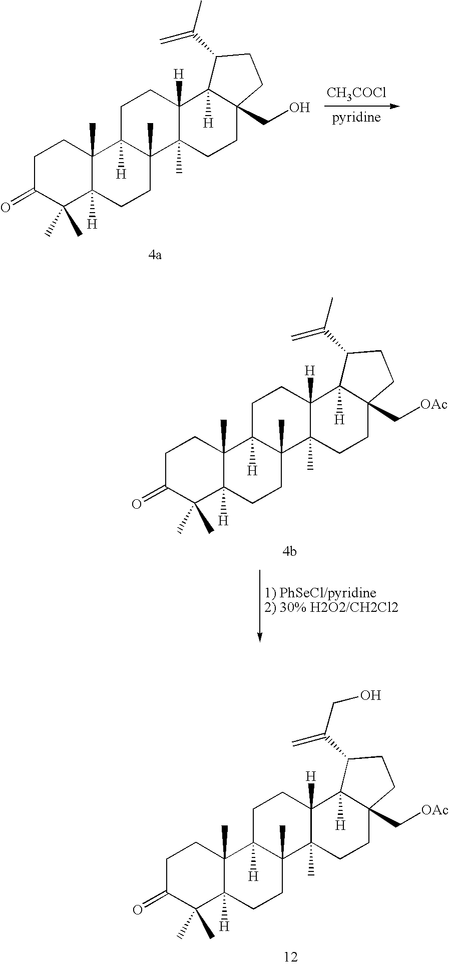

To a solution of 4a (1.8 g, 4.08 mmol) in pyridine (50 mL), while stirring at 0° C. under N₂, was added dropwise AcCl (0.7 mL, 9.84 mmol). The solution turned cloudy and precipitates were formed immediately. The ice bath was removed and stirring continued at room temperature. After 3 h, EtOAc (100 mL) and water (10 mL) were added and the heterogeneous mixture was washed with 10% HCl (10 mL). The organic layer was separated and washed with saturated NaHCO₃ (10 mL), dried (Na₂SO₄) and concentrated under reduced pressure to an oily residue, which was purified by SiO₂ column chromatography with gradient elution (0-50% EtOAc/hexanes) to afford 2.1 g (100% yield) of 4b as white foam. ¹H NMR (500 MHz, CDCl₃) δ 0.8-2.1 (m), 0.93 (s), 0.98 (s), 1.02 (s), 1.07 (s), 1.68 (s), 2.07 (s), 2.44 (m), 3.86 (d, J=11 Hz), 4.25 (dd, J=1.5, 11.5 Hz), 4.59 (m), 4.69 (d, J=2 Hz).

To a solution of benzeneselenenyl chloride (0.93 g, 4.87 mmol) in CH₂Cl₂ (30 mL), while stirring at 0° C. under N₂, was added pyridine (1.15 g, 1.2 mL, 14.58 mmol). The dark orange solution turned light yellow. The reaction solution was stirred for 0.5 h, into which a solution of 4b (1.17 g, 2.43 mmol) in CH₂Cl₂ (10 mL) was added. After 1.5 h, 30% aqueous H₂O₂ (1 mL, 9.8 mmol) was added dropwise and stirring continued for 1 h at 0° C. and then an additional hour at room temperature. The heterogeneous mixture was washed successively with 1 M HCl (1×50 mL) and saturated NaHCO₃ (1×50 mL). The organic layer was separated, dried (Na₂SO₄) and concentrated under reduced pressure to a foamy residue (0.8 g), which was purified by gradient silica gel column chromatography (10-50% EtOAc/hexanes) to give 540 mg (45% yield) of 12. ¹H NMR (500 MHz, CDCl₃) δ 0.80-1.6 (m), 0.93 (s), 1.00 (s), 1.02 (s), 1.07 (s), 1.08 (s), 1.75 (m), 1.90 (m), 2.07 (s), 2.17 (m), 2.43 (m), 2.48 (m), 3.85 (d, J=11 Hz), 4.05 (s), 4.26 (d, J=11 Hz), 5.01 (s), 5.09 (d, J=0.5 Hz); Positive ESI-MS, m/e 515.3 (M−H)⁺.

### Example 9

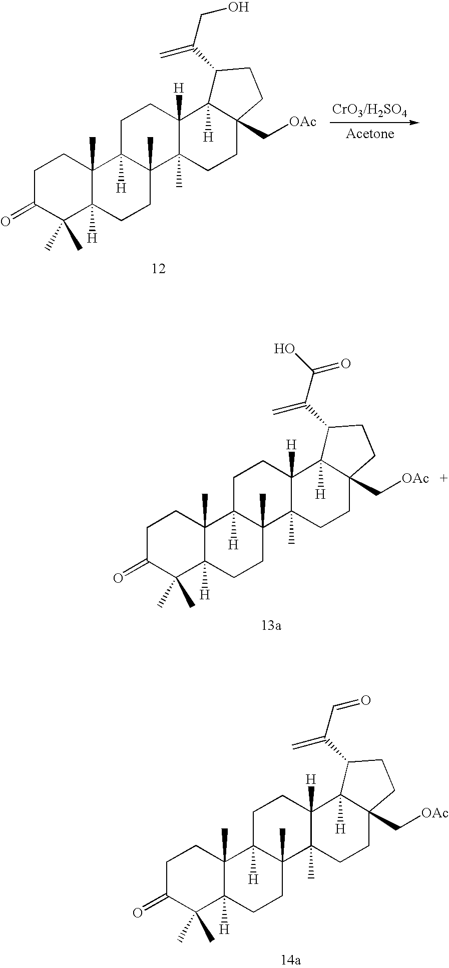

To a solution of 12 (200 mg, 0.40 mmol) in acetone (5 mL), while stirring at 0° C. under N₂, was added dropwise the freshly prepared Jones reagent (0.4 mL, 1.96 M, 0.8 mmol). After 1 h, the reaction mixture was warmed up to room temperature and stirring continued for another hour, whereupon a solution of Na₂S₂O₅ (500 mg dissolved in 15 mL of distilled water) was added. The dark green mixture was extracted with EtOAc (2×15 mL) and the combined organic layers were dried (Na₂SO₄) and concentrated under reduced pressure to a clear oil, which was purified by gradient silica gel column chromatography (10-50% EtOAc/hexanes) to afford 81 mg of 14a (41% yield) and 98 mg of 13a (48% yield). Aldehyde 14a: ¹H NMR (500 MHz, CDCl₃) δ 0.9-1.6 (m), 0.92 (s), 0.95 (s), 1.02 (s), 1.062 (s), 1.065 (s), 1.70 (m), 1.85 (m), 2.07 (s), 2.16 (m), 2.37 (m), 2.49 (m), 2.81 (m), 3.87 (d, J=10.5 Hz), 4.28 (d, J=11 Hz), 5.92 (s), 6.27 (s), 9.51 (s). Carboxylic acid 13a: ¹H NMR (500 MHz, CDCl₃) δ 0.82-2.0 (m), 0.92 (s), 0.97 (s), 1.02 (s), 1.069 (s), 1.072 (s), 2.08 (m), 2.19 (m), 2.38 (m), 2.49 (m), 2.78 (m), 3.87 (d, J=11 Hz), 4.28 (d, J=11 Hz), 5.68 (s), 6.23 (s).

### Example 10

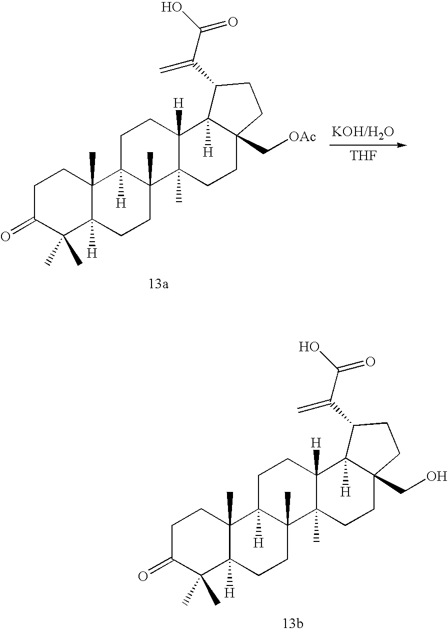

To a solution of 13a (75 mg, 0.14 mmol) in THF (4 mL), while stirring at rt, was added dropwise a solution of KOH (85%) (97 mg, 1.4 mmol) in distilled H₂O (1 mL). After stirring for 3 days, the heterogeneous mixture was diluted with EtOAc (15 mL) and washed with 1 N HCl (10 mL). The aqueous layer was extracted with EtOAc (15 mL) and the combined organic layers were dried (Na₂SO₄) and concentrated under reduced pressure. The residue thus obtained was purified by gradient silica gel column chromatography (20-100% EtOAc/hexanes) to afford 43 mg (62% yield) of 13b as a white solid. ¹H NMR (500 MHz, CDCl₃) δ 0.9-2.0 (m), 0.92 (s), 0.97 (s), 1.02 (s), 1.06 (s), 1.07 (s), 2.20 (m), 2.39 (m), 2.48 (m), 2.74 (m), 3.67 (d, J=11 Hz), 3.81 (d, J=10.5 Hz), 5.69 (s), 6.24 (s).

### Example 11

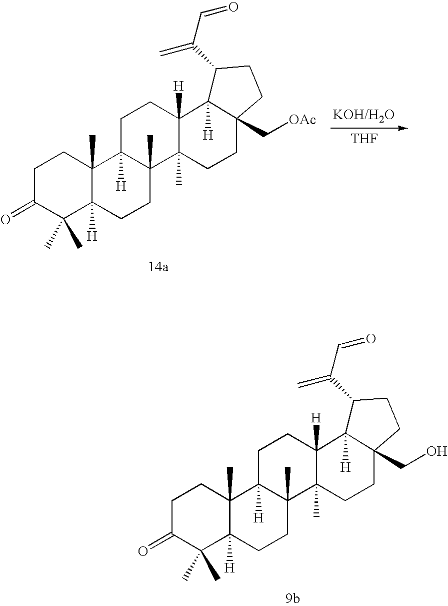

To a solution of 14a (67 mg, 0.14 mmol) in THF (4 mL), while stirring at rt, was added dropwise a solution of KOH (85%) (89 mg, 1.4 mmol) in distilled H₂O (1 mL). After 7 days of stirring, the heterogeneous mixture was diluted with EtOAc (15 mL) and washed with 1 N HCl (10 mL). The aqueous layer was extracted with EtOAc (15 mL) and the combined organic layers were dried (Na₂SO₄) and concentrated under reduced pressure. The crude product thus obtained was purified by gradient silica gel column chromatography (20-50% EtOAc/hexanes) to afford 40 mg (65% yield) of 9b as a white solid. ¹H NMR (500 MHz, CDCl₃) δ 0.8-1.6 (m), 0.91 (s), 0.96 (s), 1.02 (s), 1.04 (s), 1.07 (s), 1.69 (m), 1.87 (m), 1.94 (m), 1.99 (m), 2.18 (m), 2.38 (m), 2.48 (m), 2.76 (m), 3.38 (dd, J=4.5, 11 Hz), 3.80 (dd, J=4.5, 11 Hz), 5.93 (s), 6.28 (s), 9.50 (s).

### Example 12

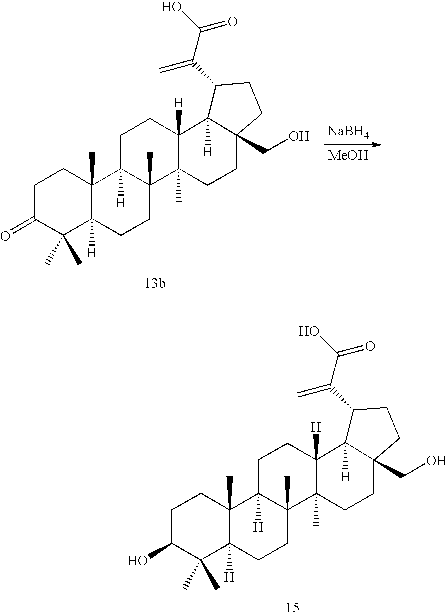

To a solution of 13b (30 mg, 0.07 mmol) in MeOH (3 mL), while stirring at room temperature under N₂, was added NaBH₄ (5 mg, 0.13 mmol). After 2 h, the reaction mixture was diluted with EtOAc (15 mL) and washed with 1 M HCl (10 mL). The aqueous layer was extracted with EtOAc (2×10 mL) and the combined organic layers were dried (Na₂SO₄) and concentrated under reduced pressure to a solid residue, which was purified by gradient SiO₂ column chromatography (50-100% EtOAc/hexanes) to afford 24 mg (77% yield) of 15 as white solid. ¹HNMR (500 MHz, CD₃OD) δ 0.68-2.1 (m), 0.75 (s), 0.85 (s), 0.95 (s), 0.98 (s), 1.07 (s), 2.16 (m), 2.73 (m), 3.11 (dd, J=5, 11.5 Hz), 3.75 (d, J=10.5 Hz), 5.58 (m), 6.06 (s).

### Example 13

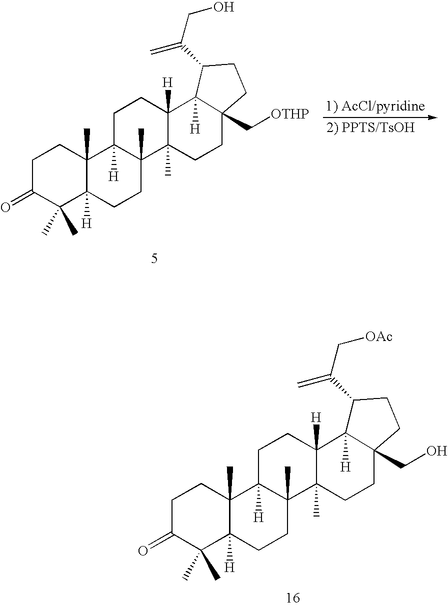

To a solution of 5 (200 mg, 0.37 mmol) in pyridine (4 mL), while stirring at 0° C. under N₂, was added dropwise AcCl (58 mg, 0.05 mL, 0.74 mmol). The reaction mixture was allowed to warm up to room temperature gradually. After 3 h, the reaction solution was partitioned between EtOAc (10 mL) and H₂O (5 mL) and acetified with 10% HCl (10 mL). The organic layer was separated, washed with saturated NaHCO₃ (50 mL), dried (Na₂SO₄) and concentrated under reduced pressure to an oily residue, which was purified by silica gel column chromatography (10-50% EtOAc/hexanes) to afford 191 mg (88% yield) of the corresponding 30-acetyloxy-28-THP protected derivative as a white foamy solid. ¹H NMR (500 MHz, CDCl₃) δ 0.8-2.2 (m), 0.92 (s), 0.98 (s), 1.02 (s), 1.05 (s), 1.07 (s), 2.10 (s), 2.40 (m), 2.46 (m), 2.98 (d, J=9.5 Hz), 3.36 (d, J=10.5 Hz), 3.51 (d, J=10 Hz), 3.53 (m), 3.83 (m), 3.91 (d, J=9.5 Hz), 4.55 (m), 4.59 (t, J=3 Hz), 4.92 (m), 4.95 (s).

The 30-acetyloxy-28-THP protected derivative obtained above (0.7 g, 1.2 mmol) was dissolved in MeOH (50 mL) at rt, into which, while stirring, were added in succession pyridinium p-toluenesulfonate (0.32 g, 1.27 mmol) and p-toluenesulfonic acid monohydrate (0.3 g, 1.57 mmol). After stirring for 0.5 h, the reaction mixture was partitioned between EtOAc (100 mL) and water (20 mL) and the resulting mixture washed with saturated NaHCO₃ (100 mL). The aqueous layer was extracted with EtOAc (150 mL) and the combined organic layers were dried (Na₂SO₄) and concentrated under reduced pressure to foamy white solid, which was further purified by SiO₂ column chromatography (15-50% EtOAc/hexanes) to afford 489 mg (82% yield) of 16 as white solid. ¹H NMR (500 MHz, CDCl₃) δ 0.9-2.2 (m), 0.92 (s), 0.99 (s), 1.02 (s), 1.06 (s), 1.07 (s), 2.10 (s), 2.31 (m), 2.41 (m), 2.43 (m), 2.46 (m), 3.32 (dd, J=5, 10.5 Hz), 3.79 (m), 4.56 (s), 4.94 (m), 4.96 (s).

### Example 14

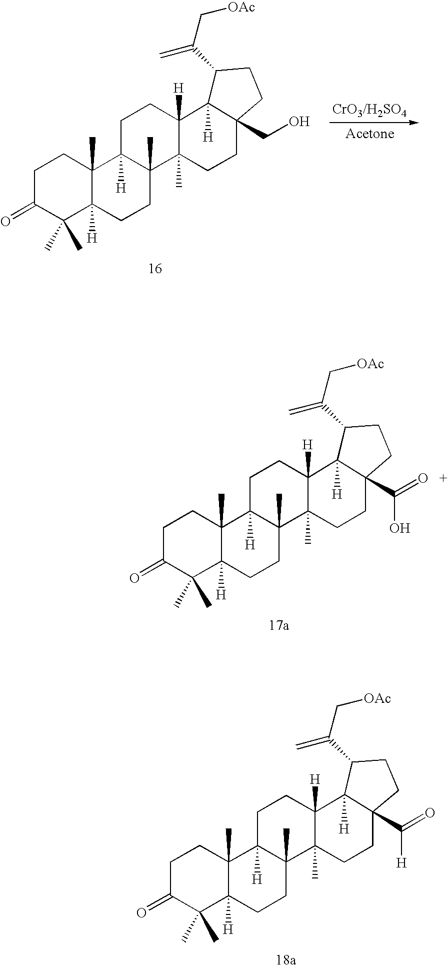

To a solution of 16 (300 mg, 0.60 mmol) in acetone (6 mL), while stirring at 0° C. under N₂, was added dropwise the freshly prepared Jones reagent (0.64 mL, 1.96 M, 1.26 mmol). When the purple-brown precipitates were formed, the reaction mixture was warmed up to room temperature and stirring continued. After 1 h, aqueous Na₂S₂O₅ solution (500 mg in 5 mL of distilled water) was added and the dark green mixture extracted with EtOAc (2×15 mL). The combined organic layers were dried (Na₂SO₄) and concentrated under reduced pressure to a clear oil which was purified by gradient silica gel column chromatography (10-50% EtOAc/hexanes) to afford 173 mg (56% yield) of 17a and 48 mg of 18a (16% yield). Acid 17a: ¹H NMR (500 MHz, CDCl₃) δ 0.82-1.6 (m), 0.92 (s), 0.97 (s), 0.99 (s), 1.01 (s), 1.07 (s), 1.71 (t, J=11.5 Hz), 1.89 (m), 1.97 (m), 2.08 (m), 2.10 (s), 2.22 (m), 2.29 (m), 2.40 (m), 2.47 (m), 2.95 (m), 4.56 (m), 4.96 (d, J=1 Hz), 4.99 (s), 9.17 (bs). Aldehyde 18a: ¹H NMR (500 MHz, CDCl₃) δ 0.8-1.6 (m), 0.92 (s), 0.95 (s), 0.98 (s), 1.01 (s), 1.07 (s), 1.78 (m), 1.89 (m), 1.98 (m), 2.08 (m), 2.10 (s), 2.40 (m), 2.47 (m), 2.84 (m), 4.56 (m), 4.97 (d, J=1.5 Hz), 5.00 (s), 9.61 (s).

### Example 15

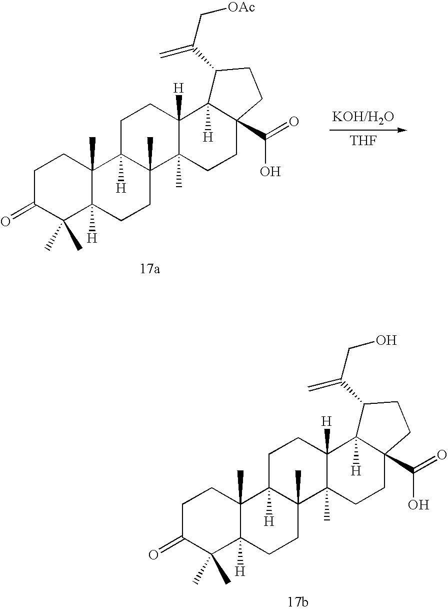

To a solution of 17a (120 mg, 0.23 mmol) in THF (4 mL), while stirring at rt, was added dropwise a solution of KOH (85%)(155 mg, 2.34 mmol) in distilled H₂O (1.0 mL). After stirring at r.t. for 16 h, the reaction mixture was diluted with EtOAc (15 mL) and washed with 1 N HCl (10 mL). The aqueous layer was extracted with EtOAc (15 mL) and the combined organic layers were dried (Na₂SO₄) and concentrated under reduced pressure. The crude product obtained was purified by gradient silica gel column chromatography (20-50% EtOAc/hexanes) to afford 85 mg (77% yield) of 17b as a white solid. ¹H NMR (500 MHz, CDCl₃) δ 0.8-1.6 (m), 0.92 (s), 0.97 (s), 0.99 (s), 1.01 (s), 1.07 (s), 1.76 (t, J=12 Hz), 1.89 (ddd, J=4.5, 7, 12.5 Hz), 1.98 (dd, J=7.5, 12.5 Hz), 2.1 (m), 2.21 (dt, J=4, 13 Hz), 2.29 (m), 2.40 (m), 2.47 (m), 2.89 (dt, J=4.5, 11 Hz), 4.13 (m), 4.93 (s), 4.98 (m).

### Example 16

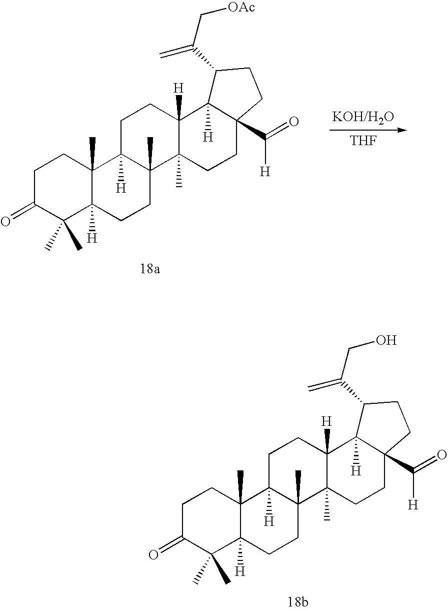

To a solution of 18a (36 mg, 0.07 mmol) in THF (4 mL), while stirring at rt, was added dropwise a solution of KOH (85%) (51 mg, 0.72 mmol) in distilled H₂O (1.0 mL). After 24 h, the reaction mixture was diluted with EtOAc (10 mL) and washed with 1 N HCl (10 mL). The aqueous layer was extracted with EtOAc (2×10 mL) and the combined organic layers were dried (Na₂SO₄) and concentrated under reduced pressure. The crude product was purified by gradient silica gel column chromatography (20-50% EtOAc/hexanes) to afford 18 mg (55% yield) of 18b as a white solid. ¹H NMR (500 MHz, CDCl₃) δ 0.9-1.6 (m), 0.92 (s), 0.95 (s), 0.99 (s), 1.01 (s), 1.07 (s), 1.76 (m), 1.88 (m), 2.04 (m), 2.39 (m), 2.47 (m), 2.78 (m), 4.13 (d, J=6 Hz), 4.95 (m), 5.00 (s), 9.63 (d, J=2 Hz).

### Example 17

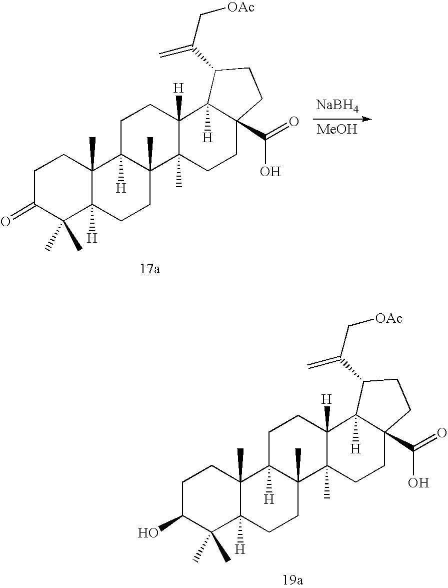

To a solution of 17a (41 mg, 0.08 mmol) in MeOH (4 mL), while stirring at room temperature under N₂, was added NaBH₄ (6 mg, 0.16 mmol). After 2 h, the reaction mixture was diluted with EtOAc (10 mL) and washed with 1 M HCl (10 mL). The aqueous layer was extracted with EtOAc (2×10 mL) and the combined organic layers were dried (Na₂SO₄) and concentrated under reduced pressure to a solid, which was purified by gradient SiO₂ column chromatography (20-50% EtOAc/hexanes) to afford 31 mg (76% yield) of 19a as white solid. ¹H NMR (500 MHz, CDCl₃) δ 0.6-1.78 (m), 0.75 (s), 0.82 (s), 0.93 (s), 0.96 (s), 0.97 (s), 1.96 (dd, J=8.5, 12.5 Hz), 2.08 (m), 2.10 (s), 2.18 (m), 2.28 (m), 2.41 (m), 2.95 (dt, J=4.5, 11.5 Hz), 3.18 (dd, J=5, 11.5 Hz), 4.56 (m), 4.95 (s), 4.98 (s).

### Example 18

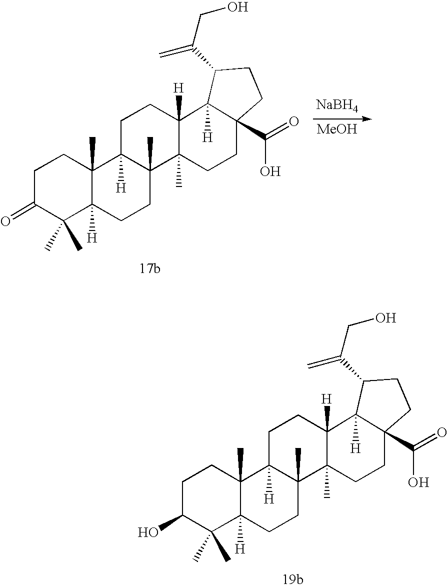

To a solution of 17b (63 mg, 0.13 mmol) in MeOH (5 mL), while stirring at room temperature under N₂, was added NaBH₄ (10 mg, 0.26 mmol). After 2 h, the reaction mixture was diluted with EtOAc (15 mL) and washed with 1 M HCl (10 mL). The aqueous layer was extracted with EtOAc (2×10 mL) and the combined organic layers were dried (Na₂SO₄) and concentrated under reduced pressure to an oily residue, which was purified by gradient SiO₂ column chromatography (20-50% EtOAc/hexanes) to afford 34 mg (54% yield) of 19b as white solid. ¹H NMR (500 MHz, CD₃OD) δ 0.68-1.7 (m), 0.74 (s), 0.85 (s), 0.94 (s), 0.96 (s), 1.01 (s), 1.74 (t, J=11 Hz), 1.88 (dd, J=8, 12.5 Hz), 2.02 (m), 2.28 (m), 2.88 (dt, J=4.5, 10.5 Hz), 3.12 (dd, J=4.5, 11 Hz), 4.04 (m), 4.87 (s), 4.96 (m).

### Example 19

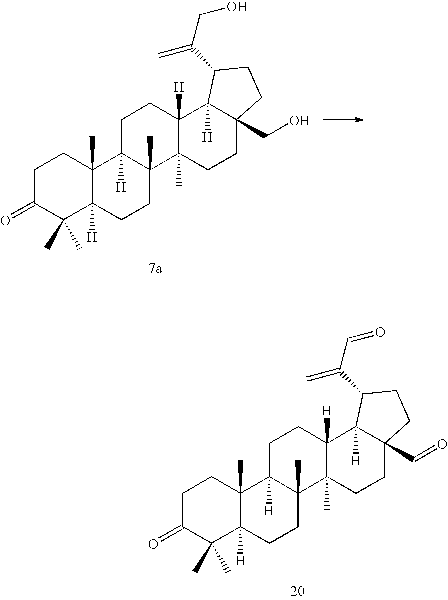

A solution of pyridine (4.54 mL, 56.22 mmol), in CH₂Cl₂ (35 mL) was stirred at room temperature under N₂ as CrO₃ (2.81 g, 28.11 mmol) was added all at once. The resulting mixture was stirred for 3 h at rt, cooled to 0° C. and a solution of 7a (1.07 g, 2.34 mmol) in CH₂Cl₂ (20 mL) was added dropwise. Stirring was continued at 0° C. for 6 h, and the reaction mixture was warmed to rt, passed through a short-path silica gel column and eluted with Hexanes/EtOAc (20:1 to 10:1) to give 0.18 g (17% yield) of 20. ¹H NMR (500 MHz, DMSO-D₆) δ 0.83 (s), 0.86 (s), 0.91 (s), 0.92 (s), 0.97 (s), 0.83-2.06 (m), 2.33 (m), 2.41 (m), 3.12 (m), 6.13 (s), 6.51 (s), 9.61 (d, J=1.5 Hz).

### Example 20

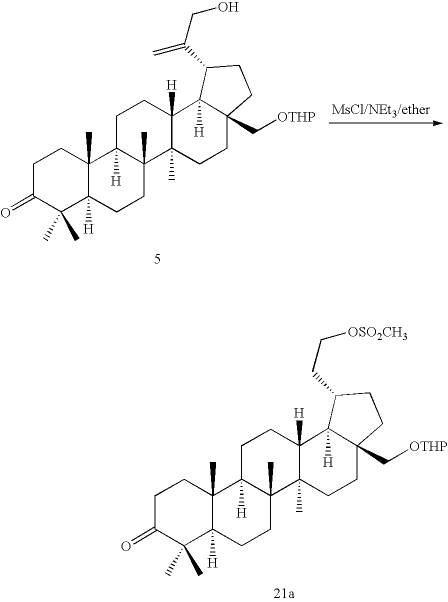

To a solution of 5 (99 mg, 0.183 mmol) and NEt₃ (0.2 mL, 1.43 mmol) in ether (4 mL), while stirring at 0° C. under N₂, was added dropwise CH₃SO₂Cl (0.05 mL, 0.64 mmol). The solution turned to light yellow and precipitates were formed. After 15 min, the reaction mixture was diluted with ether (10 mL) and washed with H₂O (10 mL). The organic layer was separated, dried (Na₂SO₄) and concentrated under reduced pressure. The crude product thus obtained was purified by silica gel column chromatography (0-100% EtOAc/hexanes and then 10% MeOH/CH₂Cl₂) to afford 113 mg (100% yield) of 21a as a mixture of diastereomers. ¹H NMR (500 MHz, CDCl₃) δ 0.8-2.2 (m), 0.92 (s), 0.99 (s), 1.024 (s), 1.026 (s), 1.061 (s), 1.07 (s), 2.40 (m), 2.47 (m), 2.98 (d, J=9.5 Hz), 3.03 (s), 3.14 (s), 3.36 (d, J=10 Hz), 3.50 (d, J=9.5 Hz), 3.54 (m), 3.83 (m), 3.91 (d, J=10 Hz), 4.54 (t, J=3 Hz), 4.58 (t, J=3 Hz), 4.67 (m), 5.08 (s).

### Example 21

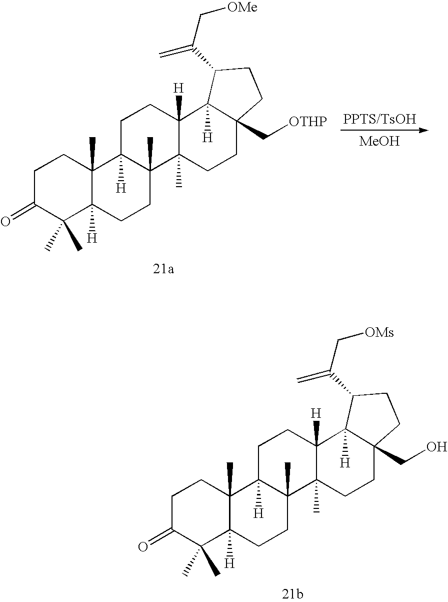

To a solution of 21a (41 mg, 0.07 mmol) in MeOH (2 mL), while stirring at rt, were added in succession pyridinium p-toluenesulfonate (20 mg, 0.08 mmol) and p-toluenesulfonic acid monohydrate (20 mg, 0.11 mmol). After 16 h, the reaction mixture was diluted with EtOAc (10 mL) and washed with saturated NaHCO₃ (10 mL). The organic layer was separated, dried (Na₂SO₄) and concentrated under reduced pressure. Purification by SiO₂ column chromatography (50% EtOAc/hexanes) afforded 20 mg (57% yield) of 21b as white solid. ¹H NMR (500 MHz, CDCl₃) δ 0.8-2.2 (m), 0.92 (s), 1.00 (s), 1.02 (s), 1.06 (s), 1.07 (s), 2.40 (m), 2.48 (m), 3.03 (s), 3.32 (m), 3.80 (m), 4.68 (m), 5.09 (s).

### Example 22

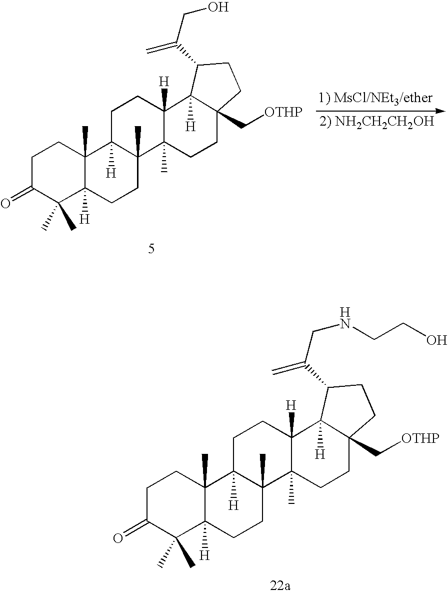

To a solution of 5 (100 mg, 0.185 mmol) and NEt₃ (0.18 mL, 1.29 mmol) in ether (4 mL), while stirring at 0° C. under N₂, was added dropwise CH₃SO₂Cl (0.05 mL, 0.64 mmol). The solution turned to light yellow and precipitates were formed. After 1 h, the reaction mixture was decanted. Into the solution was added ethanolamine (0.1 mL, 1.65 mmol). The resulting solution was heated at reflux for 4 h, whereupon it was cooled down to rt, diluted with ether (10 mL) and washed with H₂O (10 mL). The organic layer was separated, dried (Na₂SO₄) and concentrated under reduced pressure. The residue obtained was purified by silica gel column chromatography (0-100% EtOAc/hexanes and then 10% MeOH/CH₂Cl₂) to afford 27 mg (25% yield) of 22a as a mixture of diastereomers. ¹H NMR (500 MHz, CDCl₃) δ 0.9-2.2 (m), 0.92 (s), 0.98 (s), 1.023 (s), 1.025 (s), 1.05 (s), 1.07 (s), 2.03 (d, J=6 Hz), 2.39 (m), 2.47 (m), 2.84 (m), 2.98 (m), 3.00 (s), 3.24 (m), 3.37 (d, J=9.5 Hz), 3.52 (m), 3.69 (t, J=5.5 Hz), 3.79 (t, J=5 Hz), 3.84 (m), 3.92 (d, J=9.5 Hz), 4.54 (t, J=3 Hz), 4.58 (t, J=3 Hz), 4.88 (s), 4.91 (s).

### Example 23

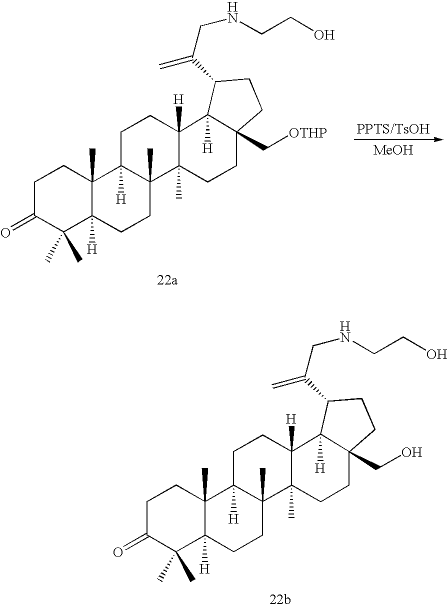

To a solution of 22a (21 mg, 0.04 mmol) in MeOH (2 mL), while stirring at rt, were added in succession pyridinium p-toluenesulfonate (10 mg, 0.04 mmol) and p-toluenesulfonic acid monohydrate (10 mg, 0.05 mmol). After 16 h, the reaction mixture was diluted with EtOAc (10 mL) and washed with saturated NaHCO₃ (10 mL). The organic layer was separated, dried (Na₂SO₄) and concentrated under reduced pressure. Purification by SiO₂ column chromatography (50% EtOAc/hex and then 10% MeOH/CH₂Cl₂) afforded 10 mg (56% yield) of 22b as white solid. ¹H NMR (500 MHz, CDCl₃) δ 0.9-2.6 (m), 0.92 (s), 0.98 (s), 1.02 (s), 1.06 (s), 1.07 (s), 2.87 (m), 2.29 (m), 3.34 (d, J=10.0 Hz), 3.72 (m), 3.77 (d, J=10.0 Hz), 4.92 (s), 4.95 (s).

### Example 24

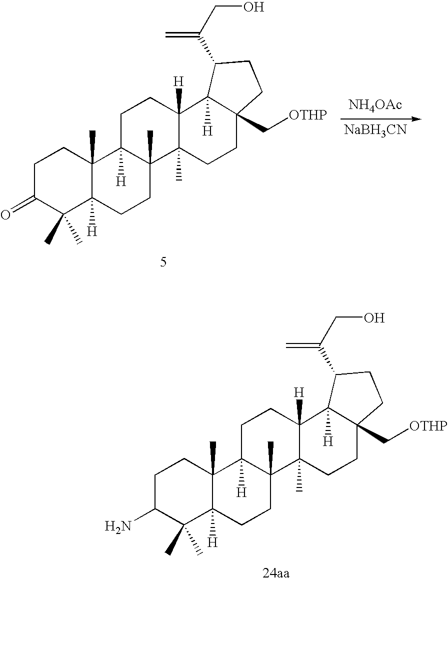

To a solution of 5 (0.3 g, 0.55 mmol) in MeOH (10 mL) at room temperature was added NH₄OAc (0.86 g, 11.09 mmol). The reaction mixture was heated to 60° C. for 1 h, whereupon it was cooled to rt, to which NaBH₃CN (0.11 g, 1.71 mmol) in MeOH (2 mL) was added. The resulting mixture was stirred at room temperature overnight, quenched with 10% NH₄OH (10 mL) and extracted with EtOAc (20 mL). The organic layer was separated, washed successively with water (15 mL) and brine (15 mL), dried (Na₂SO₄) and concentrated under reduced pressure to a crude foam solid which was purified by silica gel column chromatography (5-20% MeOH/CH₂Cl₂) to furnish 180 mg 24aa (59% yield). ¹H NMR (500 Hz, DMSO-D₆) δ 0.73 (s), 0.76 (s), 0.94 (s), 0.96 (s), 0.98 (s), 0.73-2.02 (m), 2.29 (m, 1H), 2.74 (m), 2.93 (m, 1H), 3.41 (m), 3.44 (m, 1H), 3.72 (m, 1H), 3.81 (d, J=10Hz), 3.88 (s, 2H), 4.53 (m, 1H), 4.73 (m, 2H), 4.85 (m, 1H), 7.41 (b, 3H).

### Example 25

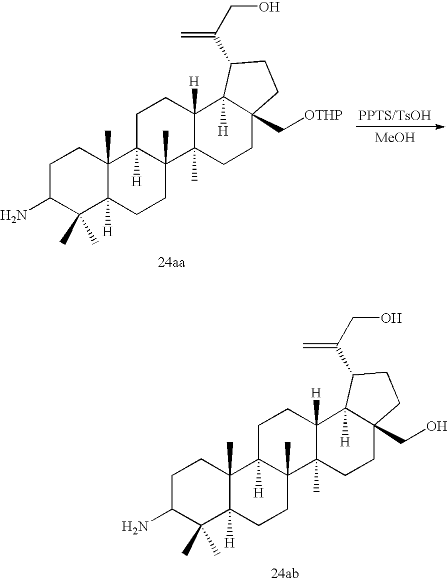

To a solution of 24aa (0.13 g, 0.23 mmol) in MeOH (3 mL), while stirring at rt, were added PPTS (0.05 g, 0.20 mmol) and TsOH·H₂O (0.05 g, 0.26 mmol). After stirring at room temperature for 24 h, the reaction mixture was diluted with EtOAc (20 mL) and washed successively with saturated NaHCO₃ (20 mL) and brine (10 mL). The organic layer was separated, dried (Na₂SO₄) and concentrated under reduced pressure. Purification by silica gel column chromatography (5-20% MeOH/CH₂Cl₂) to afford 21 mg of 24ab (19% yield), along with 18 mg of the corresponding tosylic salt of 24ab (12% yield). Compound 24ab: m.p. 189-199° C.; ¹H NMR (500 Hz, DMSO-D₆) δ 0.62 (s, 3H), 0.75 (s, 3H), 0.86 (s, 3H), 0.92 (s, 3H), 0.97 (s, 3H), 0.60-2.15 (m, 27H), 2.23 (1H), 3.06 (d, J=9 Hz, 1H), 3.51 (d, J=9 Hz, 1H), 3.87 (s, 2H), 4.19 (s, (1H), 4.74 (m, 2H), 4.84 (d, J=1.5 Hz, 1H). Positive ESI-MS, m/e 459.7 (M−2H)₂⁺. Tosylic salt of 24ab: m.p. 255° C. charred; ¹H NMR (500 Hz, DMSO-D6) δ 0.78-1.97 (m, 43H), 2.23 (m, 1H), 2.28 (s, 3H), 3.05 (m, 1H), 3.50 (m, 1H), 3.88 (m, 2H), 4.20 (m, 1H), 4.71 (m, 1H), 4.74 (s, 1H), 4.84 (d, J=1.5 Hz, 1H), 7.11 (dd, J=1, 1.5 Hz, 2H), 7.48 (dd, J=2, 1.5 Hz, 2H).

### Example 26

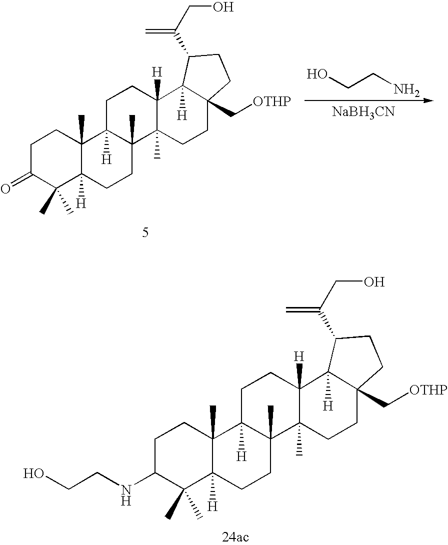

To a solution of 5 (0.4 g, 0.74 mmol) in MeOH (7 mL) at room temperature was added ethanolamine (0.13 g, 2.21 mmol). After stirring at room temperature for 0.5 h, NaBH₃CN (0.18 g, 2.95 mmol) in MeOH (2 mL) and NaOAc (0.22 g, 2.77 mmol) were added. The resulting reaction mixture was stirred at room temperature for 15 days, whereupon it was quenched with 10% NH₄OH (5 mL) and extracted with EtOAc (2×40 mL). The combined organic layer was washed with brine (25 mL), dried (Na₂SO₄) and concentrated under reduced pressure to a crude solid, which was purified by silica gel column chromatography (10-100% EtOAc/hexanes) to produce 53 mg (12% yield) of 24ac: m.p. 237-240° C.; ¹H NMR (500 MHz, DMSO-D₆) δ 0.63 (s, 3H), 0.76 (s, 3H), 0.90 (s, 3H), 0.94 (s, 3H), 0.97 (s, 3H), 0.63-1.98 (m), 2.29 (m, 1H), 2.40 (m, 1H), 2.73 (m, 1H), 2.93 (d, J=9 Hz), 3.29 (m), 3.42 (m, 4H), 3.71 (m, 1H), 3.81 (d, J=9.5 Hz), 3.88 (s, 2H), 4.36 (b, 1H), 4.53 (m), 4.76 (m, 2H), 4.85 (d, J=2 Hz).

### Example 27

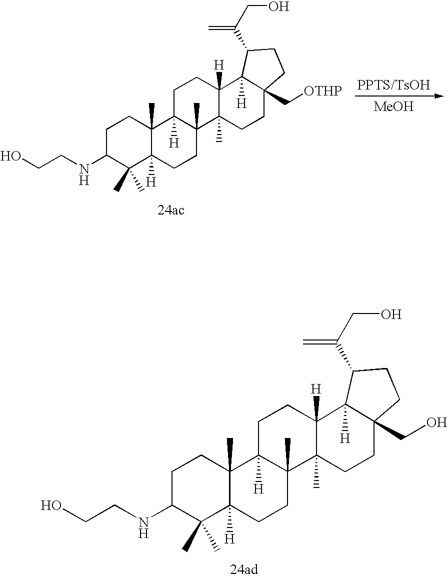

To a solution of 24ac (0.04 g, 0.06 mmol) in MeOH (3 mL), while stirring at rt, were added PPTS (0.02 g, 0.05 mmol) and p-TsOH·H₂O (0.01 g, 0.07 mmol). After 48 h, the reaction mixture was diluted with EtOAc (5 mL) and washed successively with saturated NaHCO₃, water (3 mL) and brine (5 mL). After dried (Na₂SO₄) and concentrated under reduced pressure, the crude solid was purified by silica gel column chromatography (10-100% EtOAc/hexanes) to furnish 16 mg (51% yield) of 24ad: m.p. 192-198° C.; ¹H NMR (500 Hz, DMSO-D₆) δ 0.64 (s), 0.77 (s), 0.92 (d, J=5 Hz), 0.98 (s), 0.64-1.98 (m), 2.22 (m), 2.47 (m), 2.78 (m), 3.06 (d, J=10 Hz), 3.43 (t, J=6, 5.5 Hz), 3.51 (d, J=10 Hz), 3.87 (s), 4.19 (s), 4.74 (m), 4.84 (d, J=2 Hz). Positive APCI-MS, m/e 502.25 (M−H)⁺.

### Example 28

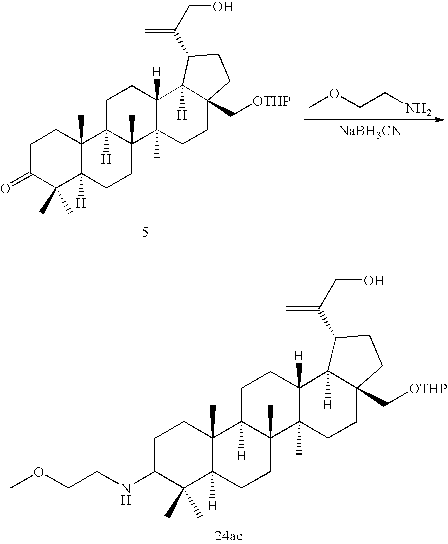

To a solution of 5 (0.5 g, 0.92 mmol) in MeOH (7 mL) at room temperature was added 2-methoxyethyl-amine (0.21 g, 2.77 mmol). After stirring at room temperature for 0.5 h, NaBH₃CN (0.23 g, 3.69 mmol) in MeOH (2 mL) and NaOAc (0.28 g, 3.46 mmol) were added. The resulting reaction mixture was stirred at room temperature for 23 days, whereupon it was quenched with 10% NH₄OH (5 mL) and extracted with EtOAc (2×40 mL). The combined organic layer was washed with brine (25 mL), dried (Na₂SO₄) and concentrated under reduced pressure to a crude solid, which was further purified by silica gel column chromatography (10-100% EtOAc/hexanes) to produce 0.16 g (29% yield) of 24ae: m.p. 111-120° C.; ¹H NMR (500 MHz, DMSO-D₆) δ 0.61 (s, 3H), 0.76 (s, 3H), 0.89 (s, 3H), 0.93 (s, 3H), 0.97 (d, J=1 Hz, 3H), 0.61-1.98 (m), 2.28 (m, 1H), 2.47 (m), 2.84 (m, 1H), 2.93 (d, J=9.5 Hz), 3.22 (s, 3H), 3.24-3.40 (m), 3.44 (m, 1H), 3.72 (m, 1H), 3.81 (d, J=9.5 Hz), 3.89 (d, J=4.5 Hz), 4.53 (m, 1H), 4.73 (m, 2H), 4.85 (d, J=2 Hz, 1H).

### Example 29

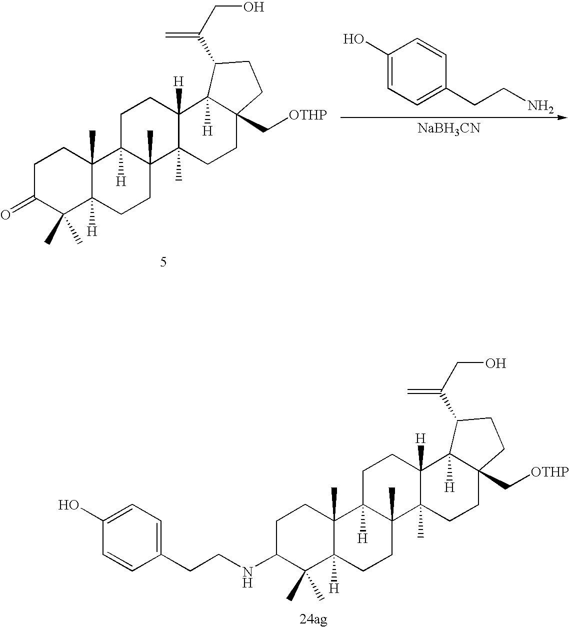

To a solution of 5 (0.50 g, 0.92 mmol) in MeOH (7 mL) at room temperature was added tyramine (0.38 g, 2.77 mmol). After stirring at room temperature for 0.5 h, NaBH₃CN (0.23 g, 3.69 mmol) in MeOH (2 mL) and NaOAc (0.28 g, 3.46 mmol) were added. The reaction mixture was stirred at room temperature for 11 days, whereupon it was quenched with 10% NH₄OH (5 mL) and extracted with EtOAc (2×40 mL). The combined organic layer was washed successively with water (10 mL) and brine (25 mL), dried (Na₂SO₄) and concentrated under reduced pressure to a crude solid, which was purified by silica gel column chromatography (10-50% EtOActo hexanes) to yield 0.16 g (26% yield) of 24ag: m.p. 135-142° C.; ¹H NMR (500 MHz, DMSO-D₆) δ 0.59 (s, 3H), 0.75 (s, 3H), 0.86 (s, 3H), 0.93 (s, 3H), 0.97 (d, J=1.5 Hz, 3H), 0.59-2.01 (m), 2.28 (m, 1H), 2.54 (m), 2.86 (m, 1H), 2.93 (m), 3.29 (m), 3.4 (m), 3.45 (m, 1H), 3.73 (m, 1H) 3.81 (m), 3.88 (s, 2H), 4.53 (m, 1H), 4.73 (m, 2H), 4.85 (d, J=10 Hz, 1H), 6.65 (d, J=10 Hz, 2H), 6.98 (d, J=8.5 Hz, 2H), 9.08 (s, 1H).

### Example 30

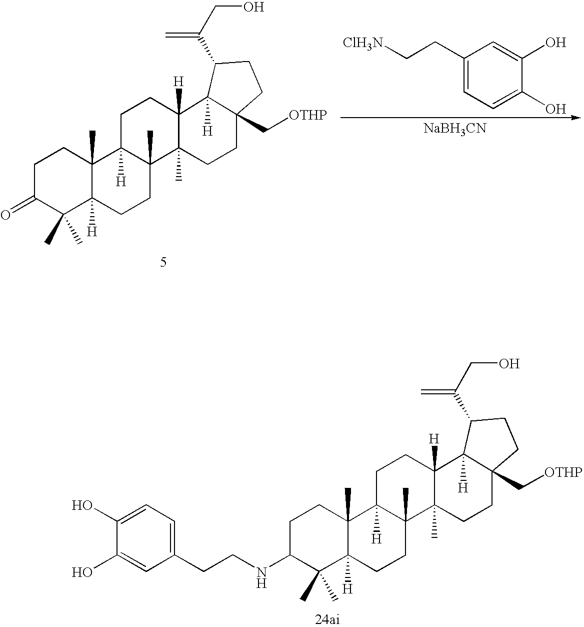

To a solution of 5 (0.4 g, 0.74 mmol) in MeOH (7 mL) at room temperature was added dopamine hydrochloric salt (0.42 g, 2.21 mmol). After stirring at room temperature for 0.5 h, NaBH₃CN (0.18 g, 2.95 mmol) in MeOH (2 mL) and NaOAc (0.22 g, 2.77 mmol) were added and the resulting reaction mixture was stirred at room temperature for 15 days, whereupon it was quenched with 10% NH₄OH (5 mL) and extracted with EtOAc (2×40 mL). The combined organic layer was washed with brine (25 mL), dried (Na₂SO₄) and concentrated under reduced pressure to a crude solid, which was purified by silica gel column chromatography (10-50% EtOActo hexanes) to afford 78 mg (15% yield) of 24ai: m.p. 138-147° C.; ¹H NMR (500 MHz, DMSO-D₆) δ 0.59 (s, 3H), 0.75 (s, 3H), 0.86 (s, 3H), 0.93 (s, 3H), 0.97 (d, J=1, Hz, 3H), 0.59-1.98 (m), 2.50 (m), 2.83 (m, 1H), 2.93 (d, J=9.5 Hz), 3.29 (m), 3.40 (d, J=10 Hz), 3.44 (m, 1H), 3.71 (m, 1H), 3.81 (d, J=8.5 Hz), 3.88 (s, 2H), 4.52 (m, 1H), 4.76 (m, 2H), 4.85 (d, J=1.5 Hz, 1H), 6.42 (dd, J=2, 2, Hz, 1H), 6.56 (d, J=1.5 Hz, 1H), 6.59 (m, 1H), 8.62 (b, 2H).

### Example 31

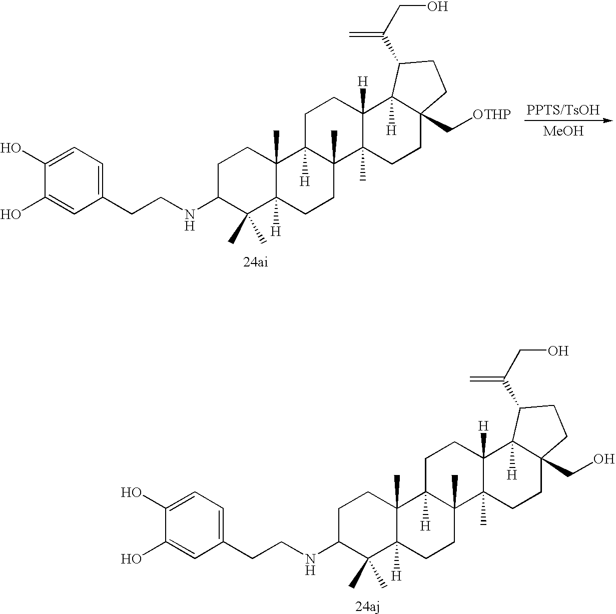

To a solution of 24ai (0.06 g, 0.09 mmol) in MeOH (3 mL), while stirring at rt, were added PPTS (0.02 g, 0.07 mmol) and p-TsOH·H₂O (0.02 g, 0.10 mmol). After 48 h, the reaction mixture was diluted with EtOAc (5 mL) and washed successively with saturated NaHCO₃, water (3 mL) and brine (5 mL). After dried (Na₂SO₄) and concentrated under reduced pressure, the crude solid obtained was purified by silica gel column chromatography (2-20% MeOH/CH₂Cl₂) to furnish 30 mg (54% yield) of 24aj: m.p. 207-214° C.; ¹H NMR (500 Hz, DMSO-D₆) δ 0.77 (s), 0.92 (s), 0.98 (s), 0.74-1.99 (m), 2.22 (m), 3.06 (m), 3.17 (d, J=5 Hz), 3.50 (m), 3.88 (s), 4.21 (t, J=5, 5 Hz), 4.71 (t, J=5.5, 5.5 Hz), 4.74 (s), 4.84 (d, J=1.5 Hz), 8.76 (b). Positive APCI-MS, m/e 594.3 (M−H)⁺.

### Example 32

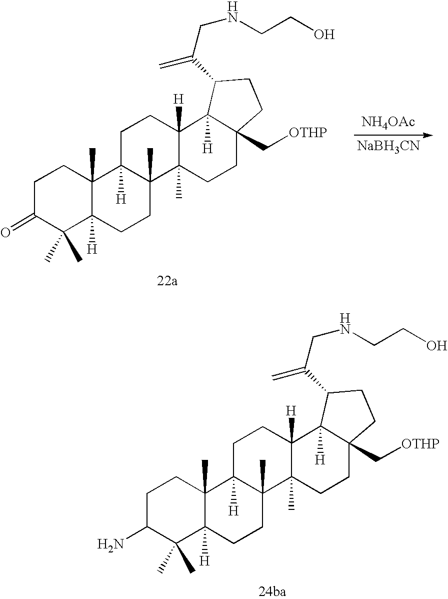

A solution of 22a (0.15 g, 0.26 mmol) and NH₄OAc (0.41 g, 5.30 mmol) in methanol anhydrous (7 mL) was heated at 60° C. for 0.5 h. The mixture was cooled to room temperature and a solution of NaBH₃CN (0.05 g, 0.82 mmol) in MeOH (2 mL) was added and the resulting reaction mixture was stirred at room temperature for 16 h days, whereupon it was quenched with 10% NH₄OH (10 mL) and water (15 mL) and extracted with EtOAc (10 mL). The organic layer was washed with brine (15 mL) and concentrated under reduced pressure to a crude material which was purification by silica gel column chromatography (5-20% MeOH/CH₂Cl₂) to afford 18 mg (12% yield) of 24ba. ¹H NMR (500 Hz, DMSO-D₆) δ 0.63 (s), 0.75 (s), 0.87 (s, 0.93 (s), 0.98 (s), 0.63-1.98 (m), 2.22 (m), 2.23 (m), 2.55 (m), 2.94 (d, J=9 Hz), 3.09 (m), 3.41 (m), 3.44 (m), 3.71 (m), 3.82 (d, J=9 Hz), 4.44 (b), 4.52 (m), 4.78 (m), 4.81 (s). Positive APCI-MS, m/e 585.3 (M−H)⁺.

### Example 33

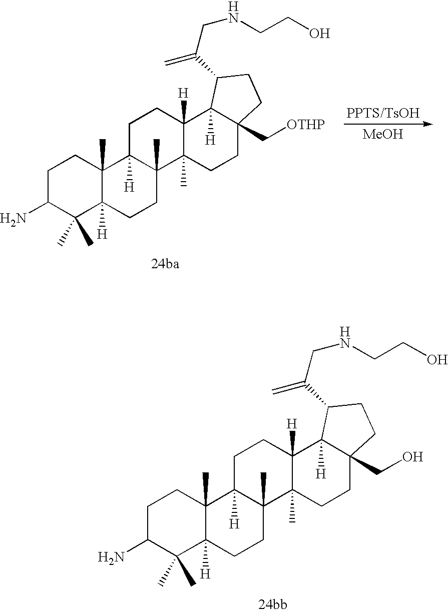

A solution of 24ba (0.08 g, 0.14 mmol) in MeOH (5 mL) was stirred at room temperature as PPTS (0.07 g, 0.28 mmol) and PTSA·H₂O (0.08 g, 0.41 mmol) were added. After stirring for 24 h, the reaction mixture was diluted with EtOAc (20 mL), and washed successively with sat. NaHCO₃, water (10 mL) brine (10 mL). The organic layer was separated, dried (Na₂SO₄) and concentrated under reduced pressure to a crude solid which was recrystallized (hexane/EtOAc) to afford 50 mg (57% yield) of 24bb (m.p. 190-203° C.). ¹H NMR (500 MHz, DMSO-D₆) δ 0.62 (s), 0.75 (s), 0.86 (s), 0.92 (s), 0.98 (s), 0.62-2.28 (m), 2.54 (m), 3.08 (m), 3.44 (m) 3.52 (m), 4.19 (b), 4.43 (b), 4.80 (d, J=5 Hz). Positive APCI-MS, m/e 501.3 (M−H)⁺.

### Example 34

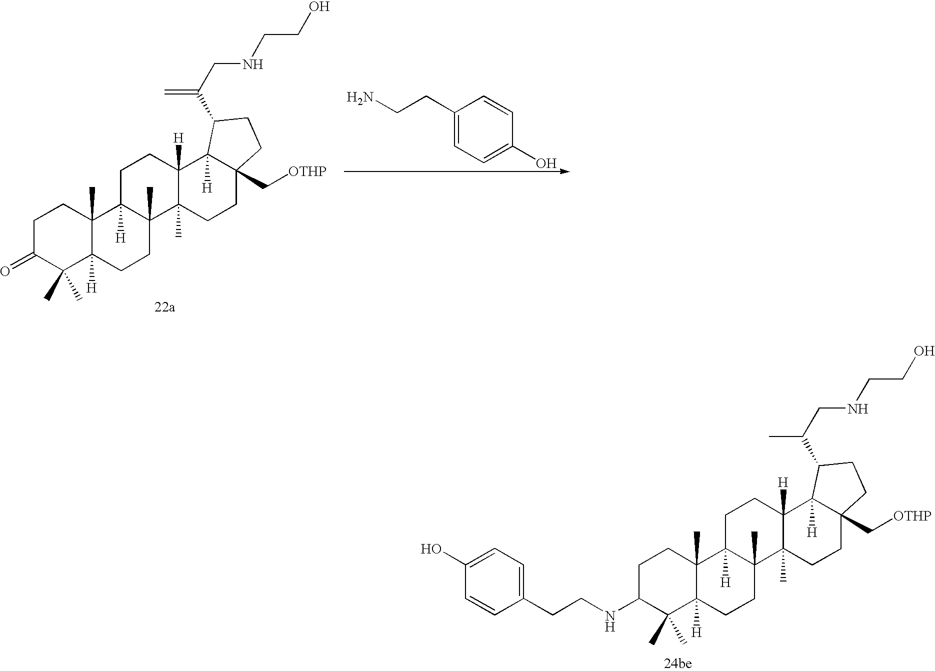

A solution of 22a (0.5 g, 0.85 mmol) and tyramine (2.35 g, 17.12 mmol) in anhydrous methanol anhydrous (7 mL) was heated at 60° C. for 4 h. After cooling to rt, a solution of NaCNBH₃ (0.42 g, 6.85 mmol) in MeOH (2 mL) and NaOAc (0.52 g, 7.5 mmol) were added. After 13 days, 10% NH₄OH (10 mL) and water (50 mL) were added and the mixture was extracted with EtOAc (40 mL). The organic layer was washed with brine (50 mL) and concentrated under reduced pressure to a crude material which was purified by silica gel column chrotography (2% MeOH/CH₂Cl₂ to 20% MeOH/CH₂Cl₂) to give 48 mg (8% yield) of 24be (m.p. 113-122° C.); ¹H NMR (500 MHz, DMSO-D₆) δ 0.59 (s), 0.75 (s), 0.86 (s), 0.93 (s), 0.97 (s), 0.59-2.11 (m), 2.36 (m), 2.55 (m), 2.69 (m), 2.87 (m), 2.94 (m), 3.43 (m), 3.72 (m), 3.82 (d, J=9.5 Hz), 4.43 (b), 4.52 (m), 4.78 (m), 4.81 (m), 6.65 (d, J=8 Hz), 6.98 (d, J=8.5 Hz), 9.08 (s). Positive APCI-MS, m/e 705.5 (M−H)⁺.

### Example 35

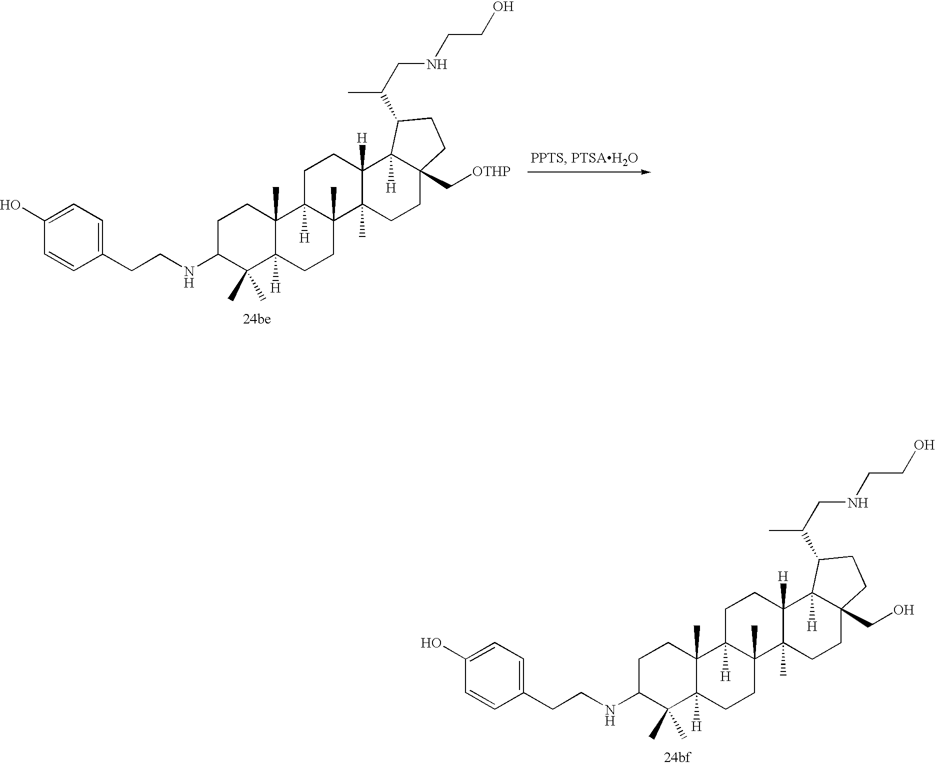

A solution of 24be (0.06 g, 0.08 mmol), PPTS (0.04 g, 0.16 mmol) and PTSA·H₂O (0.05 g, 0.25 mmol) in MeOH (3 mL) was stirred at room temperature for 24 h. The resulting solution was diluted with EtOAc (15 mL) and washed with sat. NaHCO₃, water (5 mL) and brine (10 mL). The organic layer was dried (Na₂SO₄) and concentrated under reduced pressure to a crude material which was purified by recrystallization (hexane/EtOAc) to give 4 mg (27% yield) (m.p. 145-148° C.) of 24bf. ¹H NMR (500 MHz, DMSO-D₆) δ 0.60 (s), 0.75 (s), 0.86 (s), 0.92 (s), 0.97 (s), 0.60-2.11 (m), 2.28 (b), 2.55 (m), 3.09 (m), 3.45 (d, J=6 Hz), 3.51 (m), 4.17 (t, J=5.5, 5 Hz), 4.44 (s), 4.76 (s), 4.81 (d, J=5 Hz), 6.65 (d, J=8.5 Hz), 6.98 (d, J=8 Hz), 9.09 (s). Positive APCI-MS, m/e 621.5 (M−H)⁺.

### Example 36

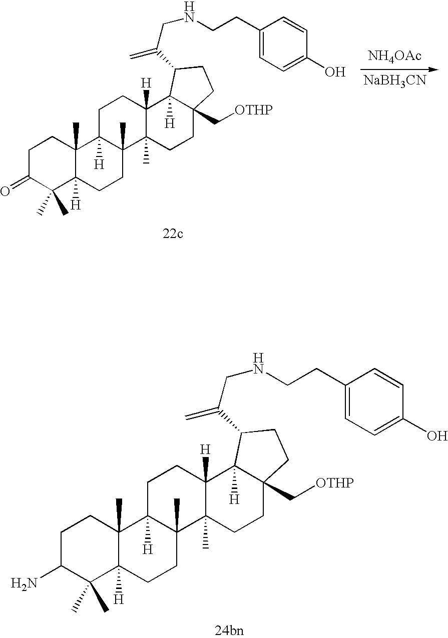

A solution of 22c (0.9 g, 1.36 mmol) and NH₄OAc (2.1 g, 27.27 mmol) in methanol anhydrous (7 mL) was heated at 60° C. for 0.5 h. The mixture was cooled to room temperature and a solution of NaBH₃CN (0.26 g, 4.22 mmol) in MeOH (3 mL) was added dropwise. After stirring at room temperature for 2 days, 10% NH₄OH (30 mL) and water (50 mL) were added and the reaction mixture was extracted with EtOAc (50 mL). The organic layer was separated, washed with brine (50 mL) and concentrated under reduced pressure to a crude material which was purified by silica gel column chromatography (2-20% MeOH/CH₂Cl₂ with 1% concn. NH₄OH) to afford 0.48 g (53% yield) of 24bn (m.p 188-193° C.). ¹H NMR (500 MHz, DMSO-D₆) δ 0.62 (s), 0.75 (s), 0.86 (s), 0.93 (s), 0.97 (s), 0.62-2.00 (m), 2.35 (b), 2.58 (m), 2.63 (m), 2.93 (d, J=9 Hz), 3.08 (m), 3.30 (m), 3.40 (d, J=69 Hz), 3.45 (m), 3.71 (m), 3.81 (d, J=9 Hz), 4.52 (m), 4.75 (s), 5.75 (s), 6.65 (m), 6.97 (d, J=8.5 Hz). D₂O-Exchange: δ 0.63 (s), 0.75 (s), 0.85 (s), 0.92 (s), 0.97 (s), 0.63-1.93 (m), 2.3 (b), 2.60 (m), 2.93 (d, J=8.5 Hz), 3.07 (s), 3.31 (d, J=9 Hz), 3.40 (d, J=9 Hz), 3.46 (s), 3.75 (m), 4.52 (s), 4.77 (d, J=17 Hz), 6.68 (d, J=8 Hz), 6.99 (d, J=8 Hz).). Positive APCI-MS, m/e 661.3 (M−H)⁺.

### Example 37

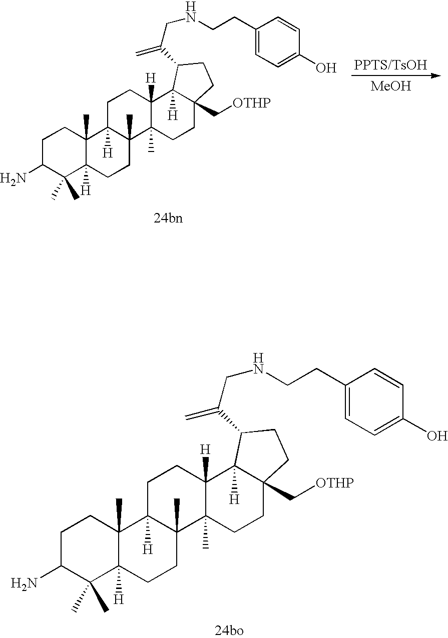

A solution of 24bn (0.27 g, 0.40 mmol) in MeOH (5 mL) was stirred at room temperature as PPTS (0.20 g, 0.81 mmol) and PTSA·H₂O (0.23 g, 1.21 mmol) were added. After stirring for 24 h, the reaction mixture was acidified with 0.1 N HCl (3 mL) and extracted with EtOAc (10 mL). The organic layer was separated, washed successively with sat. NaHCO₃ (10 mL), water (10 mL), brine (15 mL), dried (Na₂SO₄) and concentrated under reduced pressure to a crude solid which was triturated with 3:1 Hexane/EtOAc. The solid was dried under reduced pressure at 40° C. to give 0.2 g (86% yield) of 24bo (m.p. 195-200° C.). ¹H NMR (500 MHz, DMSO-D₆) [symbol] 0.65 (s), 0.75 (s), 0.89 (s), 0.92 (s), 0.98 (s), 0.65-1.98 (m), 2.31 (m), 2.62 (m), 2.65 (m), 3.08 (m), 3.51 (d, J=10.05 Hz), 4.19 (b), 4.74 (s), 6.64 (m), 6.97 (d, J=8.5 Hz). Positive ESI-MS, m/e 577.9 (M+H)⁺.

### Example 38

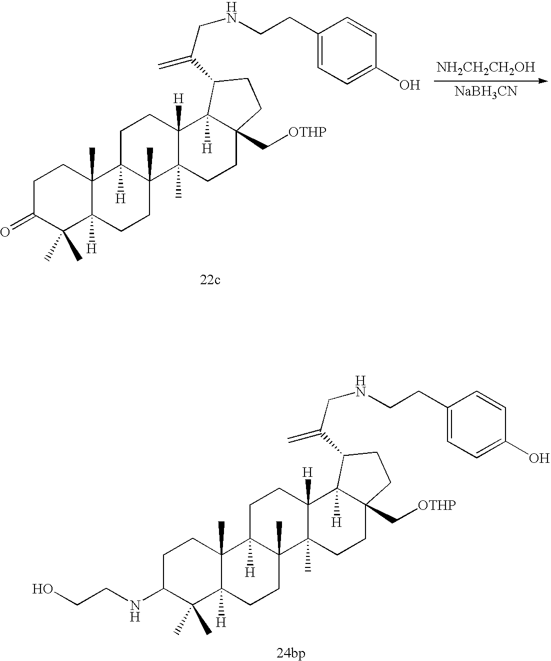

A solution of 22c (0.9 g, 1.36 mmol) in anhydrous methanol (7 mL) was stirred at room temperature under N₂ as ethanolamine (1.66 g, 27.27 mmol) was added dropwise. After stirring for 4 h, a solution of NaBH₃CN (0.69 g, 10.90 mmol) in MeOH (3 mL) and sodium acetate (0.83 g, 10.22 mmol) were added. Stirring was continued at room temperature for 4 days, whereupon the reaction was quenched with 10% NH₄OH (30 mL) and water (50 mL) and the mixture extracted with EtOAc (50 mL). The organic layer was separated, washed with brine (50 mL), and concentrated under reduced pressure to a crude material which was purified by silica gel column chromatography (2-5% MeOH/CH₂Cl₂ with 1% NH₄OH) to afford 0.5 g (52% yield) of 24 bp (m.p 156-160° C.). ¹H NMR (500 MHz, DMSO-D₆) δ 0.63 (s), 0.76 (s), 0.90 (s), 0.93 (s), 0.98 (s), 0.63-1.98 (m), 2.38 (m), 2.59 (m), 2.63 (m), 2.76 (m), 2.93 (d, J=9 Hz), 3.08 (m), 3.43 (m), 3.71 (m), 3.81 (d, J=9 Hz), 4.37 (b), 4.25 (m), 4.75 (s), 5.75 (s), 6.65 (m), 6.97 (d, J=8.5 Hz), 9.1 (s). Positive ESI-MS, m/e 705.8 (M−H)⁺.

### Example 39

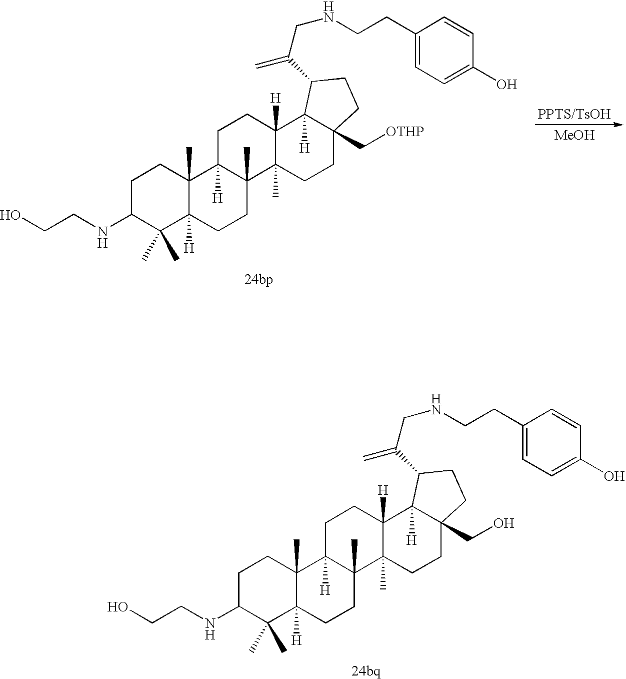

A solution of 24 bp (0.24 g, 0.33 mmol) in MeOH (5 mL) was stirred at room temperature as PPTS (0.17 g, 0.66 mmol) and PTSA·H₂O (0.2 g, 1.00 mmol) were added. After stirring for 24 h, the reaction mixture was acidified with 0.1 N HCl (3 mL) and diluted with EtOAc (10 mL). The organic layer was separated, washed with sat. NaHCO₃ (10 mL), water (10 mL), and brine (15 mL), dried (Na₂SO₄) and concentrated under reduced pressure to a crude solid which was recrystallized (EtOAc and hexane). The solid was dried under reduced pressure at 40° C. to give 0.18 g (87% yield) of 24bq (m.p. 208-212° C.). ¹H NMR (500 MHz, DMSO-D₆) δ 0.63 (s), 0.77 (s), 0.90 (s), 0.92 (s), 0.98 (s), 0.63-1.98 (m), 2.28 (b), 2.41 (m), 2.65 (m), 2.75 (m), 3.06 (m), 3.31 (s), 3.41 (t, J=5.5, 5.5 Hz), 3.51 (d, J=7 Hz), 4.19 (s), 4.37 (s), 4.74 (s), 6.65 (m), 6.97 (d, J=8.5 Hz), 9.10 (b). Positive ESI-MS, m/e 621.8 (M+H)⁺.

### Example 40

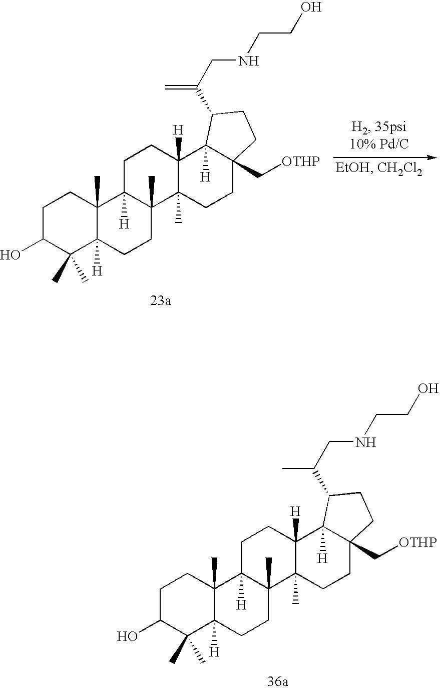

A suspension of 23a (0.15 g, 0.25 mmol) and Pd/C (10% wt on activated C) (100 mg) in 1:1 EtOH/CH₂Cl₂ (10 mL) was hydrogenated for 3 days. The suspension was filtered through celite and solids were washed with additional MeOH (100 mL). The filtrate was concentrated under reduced pressure to a foamy solid which was purified by SiO₂ column chromatography (2-10% MeOH/CH₂Cl₂) to afford 63 mg (42% yield) of 36a (m.p. 221-227° C.). ¹H NMR (500 MHz, DMSO-D₆) δ 0.66 (s), 0.78 (s), 0.88 (s), 0.94 (d, J=10.5 Hz), 0.98 (s), 0.99 (s), 0.64-2.14 (m), 2.69 (m), 2.88 (d, J=9 Hz), 2.91 (m), 2.84 (m), 3.39 (d, J=9.5 Hz), 3.68 (m), 3.79 (t, J=9.5, 10 Hz), 4.27 (m), 4.51 (m), 5.17 (b), 8.23 (b). Positive APCI-MS, m/e 588.5 (M−H)⁺.

### Example 41

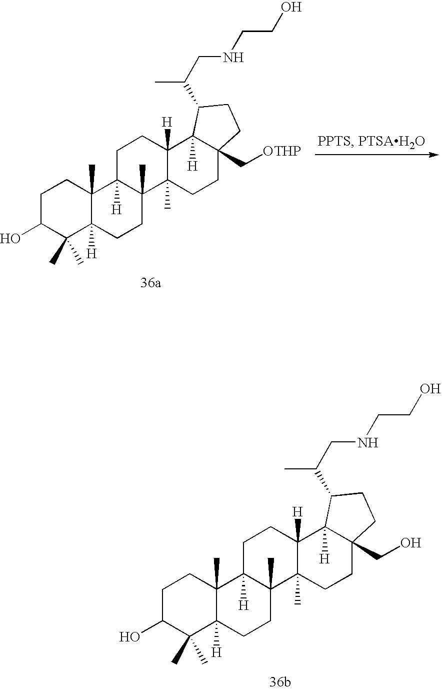

A solution of 36a (44 mg, 0.07 mmol) in MeOH (3 mL) was stirred at room temperature as PPTS (0.04 g, 0.15 mmol) and PTSA·H₂O (0.04 g, 0.22 mmol) were added. After 24 h, the reaction mixture was diluted with EtOAc (15 mL) and washed with sat. NaHCO₃, water (5 mL) and brine (10 mL). The organic layer was dried (Na₂SO₄) and concentrated under reduced pressure to a afford 29 mg (82% yield) of 36b. ¹H NMR (500 MHz, DMSO-D₆) δ 0.66 (s), 0.73 (d, J=7 Hz), 0.78 (s), 0.87 (s), 0.90 (s), 0.98 (s), 0.63-1.88 (m), 2.27 (m), 2.58 (m), 3.00 (m), 3.17 (d, J=4.5 Hz), 3.41 (m), 3.50 (m), 4.13 (m), 4.26 (d, J=5.5 Hz), 4.41 (b). Positive APCI-MS, m/e 504.3 (M−H)⁺.

### Example 42

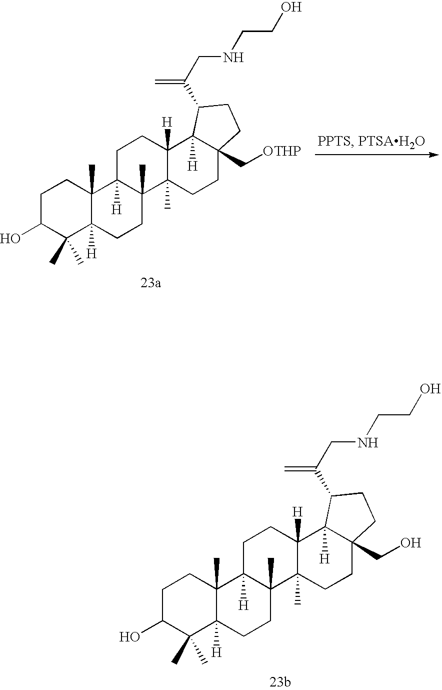

A solution of 23a (0.12 g, 0.21 mmol) in MeOH (5 mL) was stirred at room temperature as PPTS (0.11 g, 0.42 mmol) and PTSA·H₂O (0.12 g, 0.63 mmol) were added. After 24 h, the reaction mixture was diluted with EtOAc (20 mL) and washed with sat. NaHCO₃ (30 mL), water (2 mL) and brine (20 mL). The organic layer was dried (Na₂SO₄) and concentrated under reduced pressure to a crude solid which was recrystallized (hexane/EtOAc) to give 19 mg (18% yield) of 23b (m.p. 190-198° C.). ¹H NMR (500 MHz, DMSO-D₆) δ 0.65 (s), 0.76 (s), 0.87 (s), 0.92 (s), 0.97 (s), 0.62-1.98 (m), 2.28 (b), 2.55 (m), 2.96 (m), 3.07 (m), 3.45 (d, J=4.5 Hz), 3.51 (m), 4.19 (t, J=5.5, 5 Hz), 4.25 (d, J=5 Hz), 4.44 (s), 4.76 (s), 4.80 (s). Positive APCI-MS, m/e 502.3 (M−H)⁺.

### Example 43

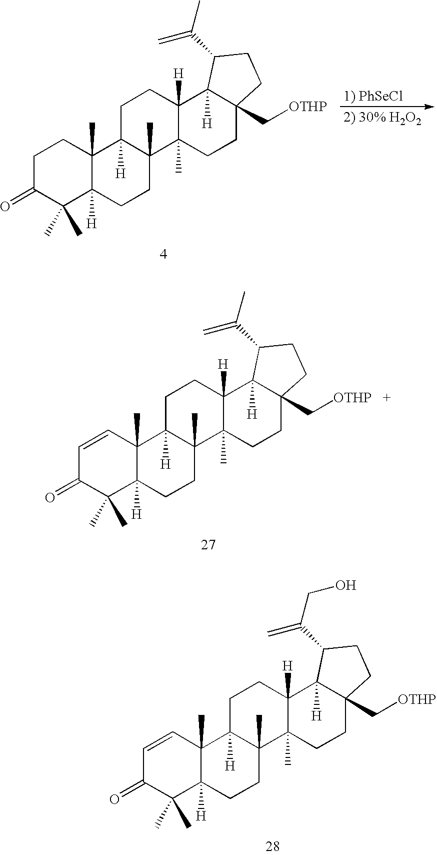

A solution of starting compound 4 (500 mg, 0.95 mmol) and benzeneselenenyl chloride (213 mg, 1.11 mmol) in EtOAc (25 mL) was stirred at room temperature for 3 h whereupon H₂O (5 mL) was added. The organic layer was separated and THF (10 mL) added. The resulting solution was treated with 30% H₂O₂ (0.76 mL, mmol) and stirred at room temperature for 1 h. The reaction mixture was then washed with saturated NaHCO₃ (30 mL) and the organic layer separated, dried (Na₂SO₄) and concentrated under reduced pressure to yield a white foam which was purified by silica gel column chromatography (5-10% EtOAc/hexanes) to afford 112 mg of 27 and 28.

### Example 44

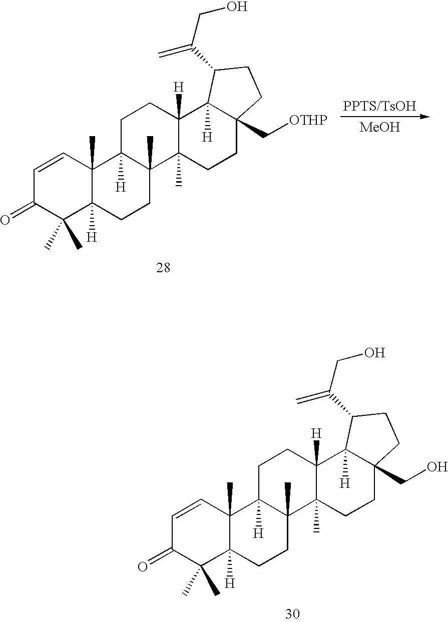

To a solution of compound 28 (53 mg, 0.10 mmol) in MeOH (2 mL), while stirring at rt, were added in succession pyridinium p-toluenesulfonate (PPTS) (20 mg, 0.08 mmol) and p-toluenesulfonic acid monohydrate (20 mg, 0.11 mmol). After 16 h, the reaction mixture was diluted with EtOAc (5 mL) and washed with saturated NaHCO₃ (5 mL). The organic layer was separated, dried (Na₂SO₄) and concentrated under reduced pressure. The crude product obtained was purified by SiO₂ column chromatography with gradient elution (10-50% EtOAc/hexane) to afford 9 mg (20% yield) of the product 30 as white solid. ¹H NMR (500 MHz, CDCl₃) δ 0.8-2.0 (m), 0.99 (s), 1.06 (s), 1.09 (s), 1.10 (s), 1.13 (s), 2.11 (m), 2.31 (m), 3.34 (m), 3.79 (m), 4.12 (m), 4.91 (m), 4.97 (m), 5.79 (d, J=10.5 Hz), 7.07 (d, J=10.0 Hz).

### Example 45

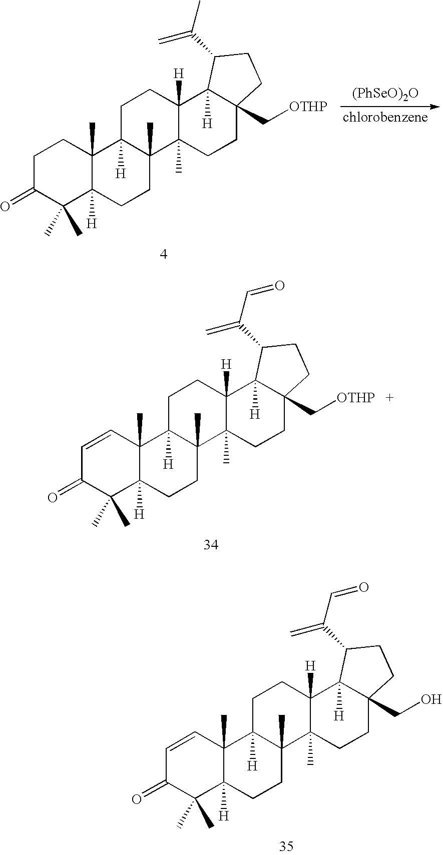

A solution of starting material 4 (200 mg, 0.38 mmol) and benzeneseleninic anhydride (70%, 275 mg, 0.53 mmol) in chlorobenzene (10 mL) was heated at reflux under N₂ for 24 h. The reaction mixture was then cooled down to rt, diluted with EtOAc (20 mL) and washed with saturated NaHCO₃ (20 mL). The organic layers were separated, dried (Na₂SO₄) and concentrated under reduced pressure to a dark yellow residue which was purified by silica gel column chromatography (0-50% EtOAc/hexanes) to afford 62 mg of 34 (30% yield) and 56 mg of 35 (33% yield). Compound 34: ¹H NMR (500 MHz, CDCl₃) δ 0.80-2.3 (m), 0.959 (s), 0.962 (s), 1.041 (s), 1.043 (s), 1.07 (s), 1.08 (s), 1.10 (s), 1.12 (s), 2.84 (m), 3.05 (d, J=10 Hz), 3.37 (d, J=10 Hz), 3.54 (m), 3.57 (d, J=9.5 Hz), 3.84 (m), 3.92 (d, J=10 Hz), 4.54 (t, J=3 Hz), 4.59 (t, J=3 Hz), 5.77 (d, J=10.5 Hz), 5.93 (s), 6.28 (s), 7.04 (d, J=10 Hz), 9.52 (s). Compound 35: ¹H NMR (500 MHz, CDCl₃) δ 0.90-2.3 (m), 0.97 (s), 1.04 (s), 1.07 (s), 1.09 (s), 1.12 (s), 2.78 (m), 3.39 (d, J=11 Hz), 3.80 (d, J=11 Hz), 5.77 (d, J=10.5 Hz), 5.94 (s), 6.29 (s), 7.04 (d, J=10.5 Hz), 9.52 (s). Positive ESI-MS, m/e 453.5 (M−H)⁺.

### Example 46

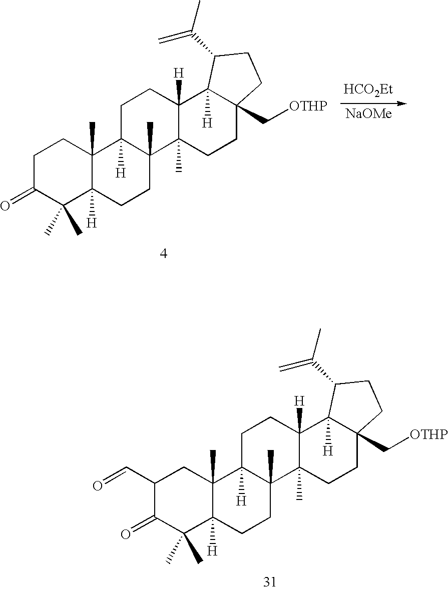

A solution of 4 (1.2 g, 2.29 mmol) in anhydrous benzene (25 mL) was stirred at room temperature under N₂ as NaOMe (0.80 g, 14.81 mmol) was added. The resulting light orange suspension was stirred for 2 h and then acidified with 0.2 N HCl (50 mL). The organic layer was washed with sat. NaHCO₃ (50 mL), dried (Na₂SO₄) and concentrated under reduced pressure to a crude material which was purified by silica gel column chromatography (0-20% EtOAc/hexanes) to afford 1.1 g (87% yield) of compound 31 as mixture of diastereomers (m.p. 150-163° C.). ¹H NMR (400 MHz, CDCl₃) δ 0.8-2.1 (m), 0.82 (s), 0.99 (s), 1.06 (s), 1.08 (s), 1.096 (s), 1.098 (s), 1.18 (s), 1.69 (s), 2.32 (d, J=14.4 Hz), 2.46 (m), 3.01 (d, J=9.6 Hz), 3.38 (d, J=9.2 Hz), 3.53 (m), 3.85 (m), 3.94 (d, J=9.6 Hz), 4.59 (m), 4.68 (s), 8.58 (d, J=2.8 Hz).

### Example 47

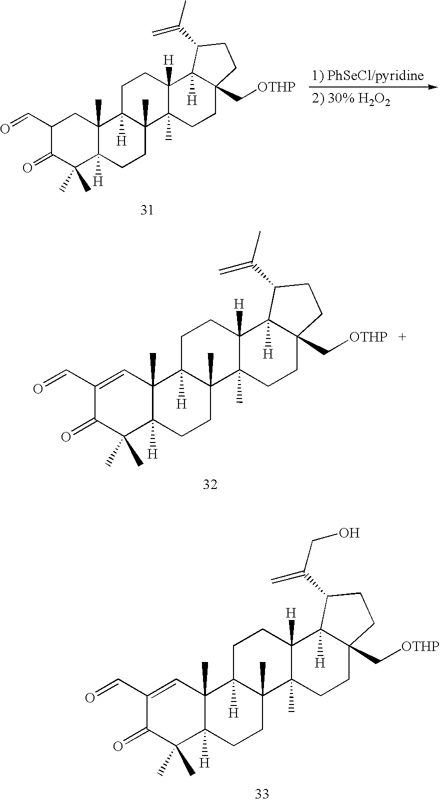

To a solution of benzeneselenenyl chloride (77 mg, 0.40 mmol) in CH₂Cl₂ (6 mL), while stirring at 0° C. under N₂, was added pyridine (0.05 mL, 0.62 mmol). The dark orange solution turned to light yellow which was stirred for 15 min at 0° C., whereupon a solution of the formyl derivative 31 (200 mg, 0.36 mmol) in CH₂Cl₂ (3 mL) was added. After stirring 1 h at 0° C., the reaction mixture was quenched with 10% HCl (10 mL). The organic layer was separated and cooled at 0° C. into which, while stirring, 30% aqueous H₂O₂ (0.1 mL, 0.98 mmol) was added dropwise. After 1 h, the reaction mixture was warmed up to room temperature and washed with saturated NaHCO₃ (10 mL). The organic layer was separated, dried (Na₂SO₄) and concentrated under reduced pressure to a foamy residue (300 mg). Purification by silica gel column chromatography using gradient (0-5% EtOAc/hexanes) provided 41 mg (21% yield) of 32 and 68 mg (33% yield) of 33 as a mixture of diastereomers. Compound 33: ¹H NMR (500 MHz, CDCl₃) δ 0.80-2.6 (m), 0.88 (s), 0.92 (s), 0.99 (s), 1.023 (s), 1.025 (s), 1.05 (s), 1.06 (s), 1.07 (s), 1.12 (s), 1.14 (s), 1.15 (s), 1.16 (s), 3.0 (d, J=9.5 Hz), 3.36 (d, J=9 Hz), 3.52 (m), 3.84 (m), 3.92 (d, J=8.5 Hz), 4.12 (m), 4.54 (t, J=3 Hz), 4.59 (t, J=3 Hz), 4.90 (m), 4.97 (m), 7.82 (d, J=2 Hz), 9.96 (s).

### Example 48

### MTS Cell Viability Assay

Cells were plated, the evening before treatment, for each treatment on 96-well plates (1×10⁴ cells/well) in 100 μL volume per well. Analog solutions were prepared by diluting each test compound in DSMO (10 mM) with the appropriate cell growth media for each individual cell line, or in “Universal Media” if several cell lines were to be assayed in parallel, immediately prior to cell treatment. Universal Media consisted of 5 mL sodium pyruvate (100× liquid stock, MediaTech), 5 mL glucose (100×, 45% liquid stock, MediaTech), 5 mL Penicillin/Streptomycin (100× liquid stock, MediaTech), 10 mL sodium bicarbonate (50× liquid stock, MediaTech), 25 mL Fetal Calf Serum, 1.25 mL insulin (4 mg/mL, Gibco) and 449 mL RPMI media with 2 mM L-glutamine for a total volume of 500 mL.

Cells were treated by aspirating plating media from each well and adding 80 μL of betulinic acid (control) or each analog solution in serial dilutions to the adherent cells. All treatments were performed in triplicate. Growth media (80 μL) was added to 3 blank wells (no cells) to measure background from the growth media. Growth media alone (no DMSO or test compound) was added to 3 wells containing cells to measure the baseline MTS activity. Vehicle (DMSO) control in serial dilutions, added to cells, was also included to monitor basal toxicity from DMSO. Cells were incubated at 37° C. for 72 h, or 120 h for the OVCAR cell lines. MTS reagent (per 96-well plate) were prepared by combining 2 mL of MTS working solution (Cell Titer AQueous Non-Radioactive Cell Proliferation Assay, Promega, cat#G1112), 100 μL of 0.92 mg/mL phenazine methosulfate/Dulbecco's PBS and 2.1 mL growth media. MTS reagent (40 μL) were added to each well and incubated at 37° C. for 1.5 to 4 h. Plates were gently shaken by hand until solution in each well appeared homogenous. Absorbances at 490 nm were measured on a Wallac Victor II plate reader at multiple time points following the addition of MTS reagent for each plate. Triplicate absorbance (490 nm) measurements were averaged following background (no cell) subtraction for each drug concentration. Percent Cell Viability was calculated for each drug concentration using the following equation:

{[Absorbance(analog treated)]/[Absorbance(DMSO treated)]}×100%

Percent viability (y-axis) was plotted against drug concentration (x-axis) and the resulting graph was used to determine the 50% inhibitory concentration (IC₅₀) for each drug.

### Example 49

### Caspase Assay

Cells were plated, the evening before treatment, for each treatment (1×10⁴ cells/well) on black-walled, clear-bottomed, 96-well plate, in 100 μL volume per well. Analog solutions were prepared immediately prior to cell treatment by diluting each test compound in the appropriate cell growth media without fetal calf serum (FCS) or “Universal Media” without FCS if several cell lines were to be assayed in parallel.

Cells were treated by aspirating plating media from each well and adding 70 μL of betulinic acid (control) or each analog solution to the adherent cells in serial dilutions. All treatments were performed in duplicate. Growth media (70 μL) was added to 2 blank wells (no cells) to measure background from the growth media. Growth media alone (no DMSO or test compound) was added to 2 wells containing cells to measure the baseline fluorescence. Vehicle (DMSO) control serial dilutions, added to cells, were also included to monitor basal caspase induction from DMSO. Cells were incubated at 37° C. for 24 h. Caspase assay reagent (per 96-well plate) was prepared according to manufacturer's instructions (Homogeneous Caspases Assay, fluorometric, Roche) by combining 6.3 mL Incubation Buffer with 0.7 mL of Substrate Stock Solution. Caspase assay reagent (70 μL) was added to each well; the plate was gently shaken by hand for 15-20 seconds and incubated at 37° C. for 4 h.

Fluorescent emission was measured at 535 nm on a plate reader using the “homogeneous caspase” program (excitation wavelength=490 nm, emission wavelength=535 nm). Duplicate wells for each treatment were averaged following background (no cells) subtraction (emission 535 nm value from all experimental emission 535 nm values) for each analog concentration. Percent change in caspase activity was calculated for each analog concentration using the following equation:

{{[Emission₅₃₅ (analog treated)]−[Emission₅₃₅ (DMSO treated)]}/Emission₅₃₅ (DMSO treated)}×100%

DMSO treatment represented baseline caspase activation in the absence of analog. The percent change in caspase activity was plotted on the y-axis for each analog treatment.

### Example 50

### Annexin-V Assay

Cells were plated (8.75×10⁵ cells/6 cm diameter tissue culture plates), the evening before treatment, in 3 mL volume per plate. This cell density is equivalent to the cell density used in the MTS and caspase assays (1×10⁴ cells/well of a 96-well plate).

Analog solutions were prepared, immediately prior to cell treatment, in the same as described for caspase assay.

Cells were treated by aspirating plating media from each well and adding 3 mL of betulinic acid (control) or each analog solution to the adherent cells in serial dilutions. Growth media alone (no DMSO or drug) was added to a plate containing cells to measure the baseline Annexin-V reactivity. Vehicle (DMSO) alone control serial dilutions were also prepared to monitor Annexin-V reactivity from DMSO. Cells were incubated at 37° C. for 6 h. Treatment media (3 mL) was removed from each plate and added to a 15 mL conical tube containing 0.333 mL FCS (final FCS concentration of 10%). The treatment media was saved to include any apoptotic/dead cells that may have detached from the plate during analog treatment. FCS was added to the recovered media to prevent further cell damage and improve the efficiency of cell pelleting during subsequent centrifugation steps (empirical observation). Adherent cells were rinsed once with PBS and 1 mL of trypsin was added. Plates were rotated several times to assure coating of the entire surface with trypsin, which was then removed. Plates were incubated at 37° C. for 4-5 min. Trypsinized cells were re-suspended in the recovered media for each sample. Cell suspension was placed back into 15 mL tubes, which was then cooled on ice. Cells were re-suspended by pipetting 7-8 times. The tubes were centrifuged at 130×g for 5 min at 4° C. The resulting cell pellets were re-suspended in 1 mL of ice cold 1× Nexin Buffer (Guava Nexin kit, Guava Technologies) and transferred to 1.5 mL conical microcentrifuge tubes to rinse any residual growth media. This procedure was followed by centrifugation of cells at 130×g for 5 min, at 4° C. Re-suspension of the resulting cell pellet in 50 μL Nexin Staining Solution (Guava Nexin kit, Guava Technologies) was followed by incubation on ice, in the dark, for 20 min. Nexin samples were analyzed immediately on the Guava flow cytometer, using the Guava Nexin software package (see Guava user's manual and Guava Nexin kit protocol on data acquisition and analysis protocols). Live cell, apoptotic cell and necrotic cell populations are differentiated by the permeability of Annexin V-PE and/or 7-AAD.

### Example 51

### Biological Results

Cytotoxicity dose response for the triterpenoid derivatives synthesized in SK-MEL-2 (melanoma), A-375 (melanoma), Daoy (glioblastoma), LN-229 (glioblastoma), OVCAR-3 (ovarian carcinoma), HT-29 (colon carcinoma), MCF-7 (breast carcinoma) cell lines using the standard MTS assay is summarized in Table 1. In Table 1, compounds 22a-d and compounds 23a-c refer to structures 22 and 23 in Scheme 5, respectively. Compounds 24aa-an and 24ba-bq refer to structure 24 in Scheme 6. Compounds 26a-e refer to structure 26 in Scheme 6. Compounds 36a-c refer to structure 36 in Scheme 10.

TABLE 1
Biological Activities of Betulinic Acid Analogs Measured as IC₅₀ (μM)

| Compound | | SK-MEL-2 | A-375 | Daoy | LN-229 | OVCAR-3 | HT-29 | MCF-7 | CEM | H-146 |
|---|---|---|---|---|---|---|---|---|---|---|
| 1 | | 7 | 22 | 11 | 29 | 40 | 31 | 30 | 2 | 8 |
| 5 | | 34 | — | 35 | 46 | 48 | 75 | 37 | — | — |
| 7a | (X = OH) | >75 | 40 | >75 | >75 | 43 | 44 | 43 | 45 | 60 |
| 8a | (X = OH) | >75 | — | >75 | — | — | — | — | — | — |
| 9a | (R = THP) | 7 | <3 | 7 | — | 9 | 9 | 32 | — | — |
| 9b | | 16 | 18 | 16 | 20 | 6 | 52 | 56 | — | — |
| 10 | | 17 | 7 | 17 | 24 | 45 | 15 | 17 | — | — |
| 11 | | >75 | — | >75 | — | — | — | — | — | — |
| 12 | | >45 | — | >45 | — | — | — | — | — | — |
| 13a | | >45 | — | >45 | — | — | — | — | — | — |
| 13b | | >75 | — | >75 | — | — | — | — | — | — |
| 14a | | 12 | 16 | 7 | 9 | 4 | 46 | 56 | — | — |
| 15 | | >75 | — | >75 | — | — | — | — | — | — |
| 16 | | 43 | — | >45 | — | — | — | — | — | — |
| 17a | | >45 | — | >45 | — | — | — | — | — | — |
| 17b | | >45 | — | >45 | — | — | — | — | — | — |
| 18a | | >45 | — | >45 | — | — | — | — | — | — |
| 18b | | >75 | — | >75 | — | — | — | — | — | — |
| 19a | | >45 | — | >45 | — | — | — | — | — | — |
| 19b | | >45 | — | >45 | — | — | — | — | — | — |
| 20 | | — | — | 34 | — | — | — | — | — | — |
| 21a | (R = CH₂OTHP) | 75 | 9 | 75 | 19 | 75 | 40 | 62 | — | — |
| 21b | (R = CH₂OH) | >75 | 21 | >75 | 46 | >75 | 17 | 54 | — | — |
| 22a | (R = CH₂OTHP, R′ = CH₂CH₂OH) | 17 | — | 21 | — | — | — | — | — | — |
| 22b | (R = CH₂OH, R′ = CH₂CH₂OH) | 45 | 10 | 47 | 16 | 67 | 45 | 47 | — | — |
| 22c | 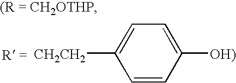 | 7 | 6 | 13 | 8 | — | 4 | 6 | — | — |
| 22d | 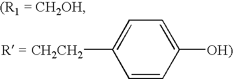 | 16 | — | 17 | — | — | — | — | — | — |
| 23a | (R = CH₂OTHP, R′ = CH₂CH₂OH) | 16 | — | 54 | — | — | — | — | — | — |
| 23b | (R = CH₂OH, R′ = CH₂CH₂OH) | 15 | — | 35 | — | — | — | — | — | — |
| 23c | 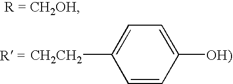 | 16 | — | 16 | — | — | — | — | — | — |
| 24aa | (R = CH₂OTHP, X = CH₂OH, R″ = H) | 5 | 0.89 | 4 | 4 | 15 | 12 | 6 | — | — |
| 24ab | (R = CH₂OH, X = CH₂OH, R″ = H) | 34 | 6 | 31 | 17 | 14 | 17 | 15 | — | — |
| 24ac | (R = CH₂OTHP, X = CH₂OH, | 15 | 13 | 15 | 11 | 7 | 60 | 36 | — | — |
| | R″ = CH₂CH₂OH) | | | | | | | | | |
| 24ad | (R = CH₂OH, X = CH₂OH, | 34 | 16 | 29 | 37 | 39 | 65 | 41 | — | — |
| | R″ = CH₂CH₂OH) | | | | | | | | | |
| 24ae | (R = CH₂OTHP, X = CH₂OH, | 34 | 6 | 28 | 17 | 17 | 19 | 16 | — | — |
| | R″ = CH₂CH₂OMe) | | | | | | | | | |
| 24af | (R = CH₂OH, X = CH₂OH, | 38 | 18 | 34 | 38 | 40 | 47 | 40 | — | — |
| | R″ = CH₂CH₂OMe) | | | | | | | | | |
| 24ag | 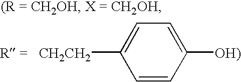 | 12 | 17 | 16 | 17 | 5 | 26 | 28 | — | — |
| 24ah | 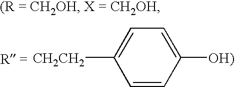 | 38 | 6 | 32 | 17 | 23 | 21 | 17 | — | — |
| 24ai | 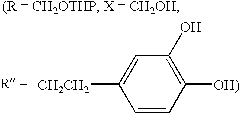 | 5 | 14 | 6 | 16 | 4 | 26 | 16 | — | — |
| 24aj | 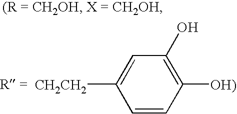 | 24 | 6 | 34 | 17 | 18 | 37 | 31 | — | — |
| 24ak | 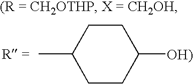 | 38 | — | 38 | — | — | — | — | — | — |
| 24al | 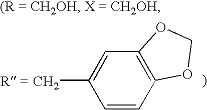 | 17 | >75 | 16 | 73 | 20 | >75 | 75 | — | — |
| 24am | R = CH₂OH, X = CH₂OH, | 16 | 17 | 16 | 17 | 2.5 | >75 | 35 | — | — |
| | R″ = CH₂CH₂NBoc) | | | | | | | | | |
| 24an | 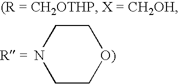 | 54 | >75 | — | — | — | — | — | — | — |
| 24ba | (R = CH₂OTHP, X = CH₂NHCH₂CH₂OH, | 5 | 3 | 5 | 15 | — | 2 | 3 | — | — |
| | R″ = H) | | | | | | | | | |
| 24bb | (R = CH₂OH, X = CH₂NHCH₂CH₂OH, | 6 | 5 | 5 | 12 | — | <2 | 2 | — | — |
| | R″ = H) | | | | | | | | | |
| 24bc | (R = CH₂OH, X = CH₂NHCH₂CH₂OH, | 8 | 4 | 5 | 28 | — | 2 | 11 | — | — |
| | R″ = CH₂CH₂OMe) | | | | | | | | | |
| 24bd | (R = CH₂OTHP, X = CH₂NHCH₂CH₂OH, | 16 | — | 16 | — | — | — | — | — | — |
| | R″ = CH₂CO₂Me) | | | | | | | | | |
| 24be | 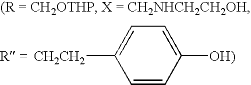 | 2 | <2 | 0.5 | 18 | — | <2 | 5 | — | — |
| 24bf | 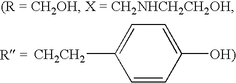 | 5 | 2 | 1 | 14 | — | <2 | 4 | — | — |
| 24bg | (R = CH₂OTHP, X = CH₂NHCH₂CH₂OH, | 5 | <2 | 5 | 18 | — | 3 | 4 | — | — |
| | R″= CH₂CH₂OH) | | | | | | | | | |
| 24bh | (R = CH₂OH, X = CH₂NHCH₂CH₂OH, | 7 | 6 | 13 | 16 | — | 2 | 6 | — | — |
| | R″ = CH₂CH₂OH) | | | | | | | | | |
| 24bi | 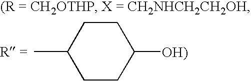 | 5 | — | 5 | — | — | — | — | — | — |
| 24bj | 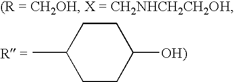 | 5 | 7 | 5 | 16 | — | 2 | 4 | — | — |
| 24bk | 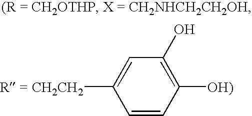 | 5 | — | 5 | — | — | — | — | — | — |
| 24bl | 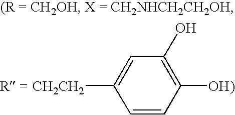 | 5 | 7 | 5 | 32 | — | 4 | 4 | — | — |
| 24bm | 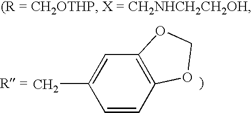 | 4 | 5 | 13 | 32 | — | 5 | 13 | — | — |
| 24bn | 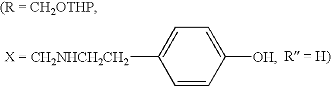 | 5 | — | 2 | — | — | — | — | — | — |
| 24bo | 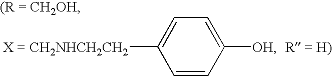 | 3 | — | <2 | — | — | — | — | — | — |
| 24bp | 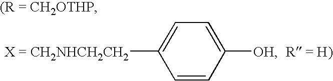 | 5 | — | <2 | — | — | — | — | — | — |
| 24bq | 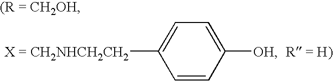 | 6 | — | 2 | — | — | — | — | — | — |
| 26a | (R₁ = OH, R = CH₂OTHP, R″ = CH₂CH₂OH) | 16 | — | 32 | — | — | — | — | — | — |
| 26b | (R₁ = OH, R = CH₂OH, R″ = CH₂CH₂OH) | 15 | — | 35 | — | — | — | — | — | — |
| 26c | 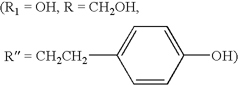 | 16 | — | 16 | — | — | — | — | — | — |
| 26d | 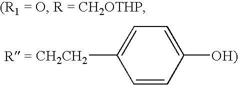 | 7 | 6 | 13 | — | — | — | — | — | — |
| 26e | 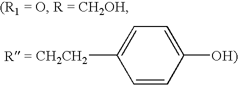 | 16 | — | 7 | 8 | — | 4 | 6 | — | — |
| 28 | | 19 | 18 | 16 | — | 30 | 19 | 5 | — | — |
| 30 | | >75 | 61 | >75 | 68 | >75 | 67 | >75 | 16 | 66 |
| 32 | | <2 | 6 | <2 | 2 | 6 | 6 | 6 | — | — |
| 34 | | 2 | 1 | 6 | 6 | 2 | 2 | 8 | 1 | 2 |
| 35 | | 14 | 6 | 17 | 58 | 6 | 8 | 32 | 2 | 5 |
| 36a | (R₁ = OH, R₂ = CH₂OTHP, | 4 | 5 | 4 | 28 | — | 5 | 7 | — | — |
| | R₃ = CH₂NHCH₂CH₂OH) | | | | | | | | | |
| 36b | (R₁ = OH, R₂ = CH₂OH, | 16 | — | 54 | — | — | — | — | — | — |
| | R₃ = CH₂NHCH₂CH₂OH) | | | | | | | | | |
| 36c | (R₁ = NHCH₂CH₂OMe, R₂ = R₃ = CH₂OH | 34 | — | 37 | — | — | — | — | — | — |

— Not tested

What is claimed is:

1. A compound of the formula 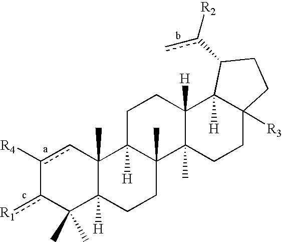
or a pharmaceutically acceptable salt thereof, wherein:
each [symbol] is independently a single or double bond;
when bond c is a double bond, then R₁ is ═O, ═S, ═N—OH, ═N—OR₅, or ═N—OCOR₅; or
when bond c is a single bond, then R₁ is H, halogen, NH₂, OH, SH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₂(R₅), OSO₂(CH₂)ₙX, OSi(R₅)ₘ(R₆)₃₋ₘ, SR₅, SCOR₅, SCO(CH₂)ₙX, SC(O)NHR₅, SC(O)NR₅R₆, NHCOR₅, NHC(O)OR₅, N(R₆)C(O)OR₅, NHC(O)NHR₅, NHC(O)NR₅R₆, N(R₆)C(O)NHR₅, or N(R₆)C(O)NR₅R₆;
R₂ and R₃ are each independently CH₂NH₂, CH₂OH, CH₂SH, CH₂SePh, CHO, CO₂H, CH₂X, CH═N—OH, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, CH₂OCO(HC═CH)ₙR₅, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NHR₅, CH₂SC(O)NR₅R₆, CH₂NHCOR₅, CH₂NHC(O)OR₅, CH₂NHC(O)NHR₅, CH₂NHC(O)NR₅R₆, CH₂N(R₆)COR₅, CH₂N(R₆)C(O)OR₅, CH₂N(R₆)C(O)NHR₅, CH₂N(R₆)C(O)NR₅R₆, CH═N—OR₅, CH═N—OCOR₅, CH(OR₅)₂, CH(OH)OR₅, CO₂R₅, CO₂NHR₅, or CO₂NR₅R₆;
R₄ is H, halogen, OH, OR₅, CN, CHO, CO₂H, CH(OR₅)₂, CH(OH)OR₅, CO₂R₅, C(O)NHR₅, or C(O)NR₅R₆;
R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein
the aryl comprises a phenyl or a polycyclic aryl group;
the heterocyle comprises a five or six membered aromatic heterocycle, a 3 to 9 membered non-aromatic heterocycle, or a polycyclic heterocycle;
wherein each alkyl, cycloalkyl, aryl and heterocycle are each optionally substituted with one or more groups which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl;
or R₅ and R₆ taken together with the carbon atoms to which they are attached form cyclo(C₃₋₉)alkyl, aryl, or heterocycle, wherein
the aryl comprises a phenyl or a polycyclic aryl group;
the heterocyle comprises a five or six membered aromatic heterocycle, a 3 to 9 membered non-aromatic heterocycle, or a polycyclic heterocycle;
wherein the cycloalkyl, aryl, and heterocycle are each optionally substituted with one or more groups which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;
each X is independently Y or aryl, heterocycle, or cyclo(C₃₋₉)alkyl, wherein the aryl, heterocycle, or cyclo(C₃₋₉)alkyl are each optionally substituted with one or more Y groups, wherein each Y is independently F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;
each m is independently 0, 1, 2, or 3;
and each n is independently 1, 2, 3, 4, or 5;
provided that
when bond a is a single bond, bond b is a double bond, R₁ is [symbol] OH or ═O, R₃ is CO₂H, and R₄ is H, then R₂ can not be CH₂OH or CHO;
when bond a is a single bond, bond b is a double bond, R₁ is [symbol] OH, R₃ is CH₂OH, and R₄ is H, then R₂ is not CH₂OH;
when bond a is a single bond, bond b is a double bond, R₁ is [symbol] OH or [symbol] OAc, R₃ is CH₂OH or CH₂OAc, and R₄ is H, then R₂ is not CH₂Br, piperidin-1′-yl, 4-methylpiperidin-1′-yl, 4-benzylpiperidin-1′-yl, 4-methylpiperazin-1′-yl, morpholin-1′-yl, cyclohexylamino, benzylamino, or sec-butylamino;
when bond a is a single bond, bond b is a double bond, R₁ is [symbol] OH or [symbol] OAc, R₃ is CO₂Me, and R₄ is H, then R₂ is not CH₂Br, or 4-methylpiperazin-1′-yl;
when bond a is a single bond, bond b is a double bond, R₁ is [symbol] OAc, R₃ is CH₂OAc, and R₄ is H, then R₂ is not O-methyl-L-valino, dodecylamino, or pentadodecylamino.

2. A compound according to claim 1, wherein R₁ is OH, ═O, O—C(O)—CH₃, NH(CH₂)₂OH, NH(CH₂)₂Cl, O-pyranyl, NH(CH₂)₂NHC(O)O-tert-butyl, NH(CH₂)₁₋₂benzodioxolyl, or NH(CH₂)₁₋₂-phenyl wherein the phenyl is substituted with OH or NH₂.

3. A compound according to claim 1, wherein R₂ is CHO, CO₂H, CH₂O-pyranyl, CH₂OH, CH₂SePh, CH₂O Ms, CH₂OAc, CH₂NHCH₂CH₂OH, CH₂NHCH₂CH₂Cl, CH₂NH(CH₂)₂—NHC(O)O-tert-butyl, CH₂NH(CH₂)₁₋₂-benzodioxolyl, or CH₂NH(CH₂)₁₋₂-phenyl wherein the phenyl is substituted with OH or NH₂.

4. A compound according to claim 1, wherein R₃ is CHO, CO₂H, CH₂O-pyranyl, CH₂OH, CH₂NHCH₂CH₂OH, CH₂NHCH₂CH₂Cl, CH₂NH(CH₂)₂—NHC(O)O-tert-butyl, CH₂NH(CH₂)₁₋₂-benzodioxolyl, or NH(CH₂)₁₋₂-phenyl, wherein the phenyl is substituted with OH or NH₂.

5. A compound according to claim 1, wherein R₄ is H, OH, CHO, CO₂H, CN, halogen, OMe.

6. A composition comprising a pharmaceutically acceptable carrier, excipient, or diluent and a compound according to the formula, 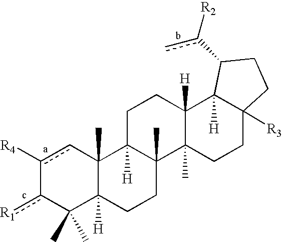
or a pharmaceutically acceptable salt thereof, wherein:
each [symbol] is independently a single or double bond;
when bond c is a double bond, then R₁ is, ═O, ═S, ═N—OH, ═N—OR₅, or ═N—OCOR₅; or
when bond c is a single bond, then R₁ is H, halogen, NH₂, OH, SH, NHR₅, NH(CH₂)ₙX, NR₅R₆, OR₅, OCOR₅, OCO(CH₂)ₙX, OC(O)OR₅, OCO(HC═CH)ₙR₅, OC(O)NHR₅, OC(O)NR₅R₆, OSO₂H, OSO₂(R₅), OSO₂(CH₂)ₙX, OSi(R₅)ₘ(R₆)₃₋ₘ, SR₅, SCOR₅, SCO(CH₂)ₙX, SC(O)NHR₅, SC(O)NR₅R₆, NHCOR₅, NHC(O)OR₅, N(R₆)C(O)OR₅, NHC(O)NHR₅, NHC(O)NR₅R₆, N(R₆)C(O)NHR₅, or N(R₆)C(O)NR₅R₆;
R₂ and R₃ are each independently CH₂NH₂, CH₂OH, CH₂SH, CH₂SePh, CHO, CO₂H, CH₂X, CH═N—OH, CH₂NHR₅, CH₂NH(CH₂)ₙX, CH₂NR₅R₆, CH₂OR₅, CH₂OCOR₅, CH₂OC(O)OR₅, CH₂OCO(CH₂)ₙX, OCO(HC═CH)ₙR₅, CH₂OC(O)NHR₅, CH₂OC(O)NR₅R₆, CH₂OSO₂(R₅), CH₂OSO₂(CH₂)ₙX, CH₂OSi(R₅)ₘ(R₆)₃₋ₘ, CH₂SR₅, CH₂SCOR₅, CH₂SCO(CH₂)ₙX, CH₂SC(O)NHR₅, CH₂SC(O)NR₅R₆, CH₂NHCOR₅, CH₂NHC(O)OR₅, CH₂NHC(O)NHR₅, CH₂NHC(O)NR₅R₆, CH₂N(R₆)COR₅, CH₂N(R₆)C(O)OR₅, CH₂N(R₆)C(O)NHR₅, CH₂N(R₆)C(O)NR₅R₆, CH═N—OR₅, CH═N—OCOR₅, CH(OR₅)₂, CH(OH)OR₅, CO₂R₅, CO₂NHR₅, or CO₂NR₅R₆;
R₄ is H, halogen, OH, OR₅, CN, CHO, CO₂H, CH(OR₅)₂, CH(OH)OR₅, CO₂R₅, C(O)NHR₅, or C(O)NR₅R₆;
R₅ and R₆ are independently a straight or branched C₁₋₈ alkyl, aryl-C₁₋₈ alkyl, heterocycle-C₁₋₈ alkyl, cyclo(C₃₋₉)alkyl, aryl, or heterocycle, wherein
the aryl comprises a phenyl or a polycyclic aryl group;
the heterocyle comprises a five or six membered aromatic heterocycle, a 3 to 9 membered non-aromatic heterocycle; or a polycyclic heterocycle;
wherein each alkyl, cycloalkyl, aryl and heterocycle are each optionally substituted with one or more groups which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I, wherein each R₇ is independently H or C₁₋₈ alkyl;
or R₅ and R₆ taken together with the carbon atoms to which they are attached form a cyclo(C₃₋₉)alkyl, aryl, or heterocycle, wherein
the aryl comprises a phenyl or a polycyclic aryl group;
the heterocyle comprises a five or six membered aromatic heterocycle, a 3 to 9 membered non-aromatic heterocycle, or a polycyclic heterocycle;
wherein the cycloalkyl, aryl and heterocycle are each optionally substituted with one or more groups which are each independently C₁₋₈ alkyl, C₁₋₈ alkoxy, C₁₋₈ alkylamino, di(C₁₋₈ alkyl)amino, C₁₋₈ alkylamino-C₁₋₈ alkyl, di(C₁₋₆ alkyl)amino-C₁₋₈ alkyl, OC(O)OR₇, OC(O)N(R₇)₂, C(O)OR₇, C(O)N(R₇)₂, S(O)₂R₇, S(O)₂N(R₇)₂, N(R₇)C(O)N(R₇)₂, N(R₇)C(O)OR₇, CN, N₃, NHOH, ═NOH, NH₂, NO₂, OH, SH, F, Cl, Br, or I wherein each R₇ is independently H or C₁₋₈ alkyl;
each X is F, Cl, Br, I, CN, N₃, NHOH, ═NOH, NH₂, OH, SH, NHR₅, NR₅R₆, OR₅, SR₅, CO₂H, CO₂R₅, SO(OH)₂, or SO(OR₅)₂;
each m is independently 0, 1, 2, or 3;
and each n is independently 1, 2, 3, 4, or 5.

7. A composition comprising a compound according to any one of claims 2-5 and pharmaceutically acceptable carrier, excipient, or diluent.

8. A method for inhibiting cancer in a cell comprising contacting the cell in which inhibition is desired with an effective amount of a compound according to claim 1 or a composition according to claim 6 or 7.

9. A method of treating a disease comprising administering to a patient a composition according to claim 6 or 7.

10. The method according to claim 9, wherein the disease involves a cell proliferative condition.

11. The method according to claim 10, wherein the cell proliferative condition is cancer.

12. The method according to claim 11, wherein the cancer is melanoma, glioblastoma, ovarian carcinoma, colon carcinoma, and breast carcinoma, or cervical cancer.

13. A method for inhibiting viruses, bacteria or malaria in a cell comprising contacting the cell in which inhibition is desired with an effective amount of a compound according to claim 1 or a composition according to claim 6 or 7.

14. A method for treating inflammation comprising administering to a patient a composition according to claim 6 or 7.
